(19) United States
(12) Patent Application Publication
(10) Pub. No.: US 2017/0253933 A1
(43) Pub. Date: Sep. 7, 2017

(54) COMPOSITIONS AND METHODS FOR TREATING AND DIAGNOSING CHEMOTHERAPY-RESISTANT CANCERS

(71) Applicant: Genentech, Inc., South San Francisco, CA (US)

(72) Inventor: Yulei WANG, South San Francisco, CA (US)

(21) Appl. No.: 15/597,789

(22) Filed: May 17, 2017

Related U.S. Application Data
(63) Continuation of application No. PCT/US2015/067427, filed on Dec. 22, 2015.
(60) Provisional application No. 62/200,340, filed on Aug. 3, 2015.
(60) Provisional application No. 62/096,355, filed on Dec. 23, 2014.

(51) Int. Cl. C12Q 1/68; A61K 39/395; C07K 16/18; A61K 45/06; C07K 16/22; A61K 31/337; A61K 31/495; A61K 31/4745; A61K 33/24; A61K 38/21; G01N 33/574; A61K 31/7068

(52) U.S. Cl. CPC C12Q 1/6886; G01N 33/57449; A61K 39/3955; C07K 16/18; A61K 45/06; A61K 31/7068; A61K 31/337; A61K 31/495; A61K 31/4745; A61K 33/24; A61K 38/212; C07K 16/22; C12Q 2600/106; C12Q 2600/112; C07K 2317/24

(57) ABSTRACT

The invention provides methods of using expression levels of one or more stroma signature genes as selection criteria for determining a patient with cancer that is chemotherapy-resistant who may benefit from a particular anti-cancer therapy, such as stroma-targeted therapy, anti-angiogenic therapy, and/or immunotherapy. The present invention also provides methods of using expression levels of one or more stroma signature genes as a selection criterion for treating cancer patients, such as ovarian cancer patients, with a stroma-targeted agent.

### SEQUENCE LISTING

The instant application contains a Sequence Listing which has been submitted electronically in ASCII format and is hereby incorporated by reference in its entirety. Said ASCII copy, created on May 16, 2017, is named 50474-092003_Sequence_Listing_5.16.17_ST25 and is 4,567 bytes in size.

### FIELD OF THE INVENTION

The present invention is directed to methods for identifying patients with chemotherapy-resistant cancer.

### BACKGROUND OF THE INVENTION

Epithelial ovarian cancer (EOC) is the leading cause of death for gynecologic malignancies, and treatment of EOC continues to present a significant clinical challenge. A current standard of care for EOC consists of aggressive surgical cytoreduction followed by adjuvant platinum- and taxane-based chemotherapy. Although response rates to this treatment are high, 20-30% of cases are resistant and progress during or within six months of completion of primary therapy. Patients with resistant cancer thus gain little benefit from this treatment and represent a significant unmet clinical need. In order to predict response to chemotherapy, and to develop novel strategies to overcome primary chemotherapy-resistance in EOC, and in cancer in general, a better understanding of molecular characteristics of chemotherapy-resistance is needed.

Activation of the host stromal microenvironment, commonly referred to as the “reactive stroma,” has been implicated as a critical component of cancer progression in many types of cancers. Stromal activation in cancer resembles the wound healing process in normal tissues, as activated stromal cells exhibit elevated production of extracellular matrix (ECM) components, growth factors, and matrix remodeling enzymes to create a tumor microenvironment that promotes cancer cell survival, proliferation, and invasion. In particular, the tumor microenvironment has been increasingly recognized to play an important role in the pathogenesis of EOC. However, the key regulators of the reactive stroma and the specific mechanisms through which the reactive stroma affects tumor progression, treatment response, and clinical outcomes in EOC are poorly understood.

Accordingly, there is a need for methods of determining whether patients are likely to respond to chemotherapeutic-based therapies, and also to develop alternative strategies for the treatment of cancer in general.

### SUMMARY OF THE INVENTION

In one aspect, the invention features methods of identifying patients with cancer that is chemotherapy-resistant, the methods including: a) determining the expression level of one or more stroma signature gene(s) in a sample obtained from a patient, b) comparing the expression level of the one or more stroma signature gene(s) to the median level of expression for the one or more stroma signature gene(s) in the cancer type, and c) determining if the patient's cancer is chemotherapy-resistant, wherein expression of the one or more stroma signature gene(s) in the patient sample at a level more than the median level for expression of the one or more stroma signature gene(s) in the cancer type indicates that the patient has cancer that is chemotherapy-resistant, e.g., in the case of detecting expression levels of one or more stroma signature genes that are up-regulated in chemotherapy (e.g., platinum-based chemotherapy)-resistant cancer. Detection of decreased levels of expression (e.g., a level less than the median level) can also indicate that the patient has cancer that is chemotherapy-resistant, in the case of detecting expression levels of one or more stroma signature genes that are down-regulated in chemotherapy (e.g., platinum-based chemotherapy)-resistant cancer.

In one embodiment, the patient has cancer that is chemotherapy-resistant if the patient's cancer has been determined to express the one or more stroma signature gene(s) at a level that is more than the 75^{th} percentile for the one or more stroma signature gene(s) expression in the cancer type (e.g., in the case of one or more stroma signature genes that are up-regulated in chemotherapy (e.g., platinum-based chemotherapy)-resistant cancer). In certain other embodiments of the above aspect, the cancer that is chemotherapy-resistant is cancer that is platinum-resistant.

In certain embodiments, the methods further include the step of identifying the patient as likely to benefit from administration of a VEGF antagonist when the patient is determined to have cancer that is chemotherapy-resistant. In certain other embodiments, the methods further include the step of administering a VEGF antagonist in a therapeutically effective amount to the patient, if the patient is determined to have cancer that is chemotherapy-resistant. In preferred embodiments, the VEGF antagonist is an anti-VEGF antibody. Preferably, the anti-VEGF antibody is bevacizumab.

In other embodiments, the methods further include the step of identifying the patient as likely to benefit from a stroma-targeted therapy when the patient is determined to have cancer that is chemotherapy-resistant. In yet other embodiments, the methods further include the step of administering a stroma-targeted agent in a therapeutically effective amount to the patient, if the patient is determined to have cancer that is chemotherapy-resistant.

In another embodiment, the methods further include the step of identifying the patient as likely to benefit from an immunotherapy when the patient is determined to have cancer that is chemotherapy-resistant. In yet another embodiment, the methods further include the step of administering an immunomodulatory agent in a therapeutically effective amount to the patient, if the patient is determined to have cancer that is chemotherapy-resistant. In preferred embodiments, the immunomodulatory agent includes a TDO2, CD36, GZMK, CD247, CD1C, CSF1, IDO1, IL7R, or CCR7 antagonist.

In a second aspect, the invention features methods of identifying patients with cancer that is chemotherapy-sensitive, the methods including: a) determining the expression level of one or more stroma signature gene(s) in a sample obtained from a patient, b) comparing the expression level of the one or more stroma signature gene(s) to the median level of expression for the one or more stroma signature gene(s) in the cancer type, and c) determining if the patient has cancer that is chemotherapy-sensitive, wherein expression of the one or more stroma signature gene(s) in the patient sample at a level less than the median level for expression of the one or more stroma signature gene(s) in the cancer type indicates that the patient has cancer that is chemotherapy-sensitive (e.g., in the case of one or more stroma signature genes that are up-regulated in chemotherapy (e.g., platinum-based chemotherapy)-resistant cancer).

In certain embodiments, the patient has cancer that is chemotherapy-sensitive if the patient's cancer has been determined to express the one or more stroma signature gene(s) at a level that is less than the 25^{th} percentile for the one or more stroma signature gene(s) expression in the cancer type. In other embodiments, the method includes the step of administering one or more chemotherapeutic agent(s) in a chemotherapy regimen, if the patient is determined to have cancer that is chemotherapy-sensitive.

In certain embodiments of the above aspects and embodiments, the sample is a tumor tissue sample. In particular embodiments, the methods are carried out prior to administering a chemotherapeutic agent in order to provide a pre-administration diagnosis. In certain embodiments, the patient has not undergone chemotherapy or the patient is currently undergoing chemotherapy.

In a third aspect, the invention features methods of identifying patients suffering from cancer who may benefit from administration of a VEGF antagonist or an immunomodulatory agent, the methods including: a) determining the expression level of one or more stroma signature gene(s) in a sample obtained from a patient, wherein expression of the one or more stroma signature gene(s) at a level more than the median level for expression of the one or more stroma signature gene(s) in the cancer type indicates that the patient may benefit from administration of a VEGF antagonist or immunomodulatory agent (e.g., in the case of one or more stroma signature genes that are up-regulated in chemotherapy (e.g., platinum-based chemotherapy)-resistant cancer), and optionally b) administering the VEGF antagonist or immunomodulatory agent in a therapeutically effective amount to the patient.

In particular embodiments, the above methods further include the step of administering one or more chemotherapeutic agents in a chemotherapy regimen. In some embodiments, the chemotherapeutic agent(s) is selected from the group consisting of a HER antibody, an antibody directed against a tumor associated antigen, an anti-hormonal compound, a cardioprotectant, a cytokine, an EGFR-targeted drug, an anti-angiogenic agent, a tyrosine kinase inhibitor, a COX inhibitor, a non-steroidal anti-inflammatory drug, a farnesyl trasferase inhibitor, an antibody that binds oncofetal protein CA 125, a Her2 vaccine, a HER targeting therapy, a raf or ras inhibitor, liposomal doxorubicin, topotecan, taxane, dual tyrosine kinase inhibitor, TLK286, EMD-7200, a medicament that treats nausea, a medicament that prevents or treats skin rash or standard acne therapy, a medicament that treats or prevents diarrhea, a body temperature-reducing medicament, and a hematopoietic growth factor. In other embodiments, the one or more chemotherapeutic agent(s) is gemcitabine, carboplatin, oxaliplatin, irinotecan, fluoropyrimidine (e.g., 5-FU), paclitaxel (e.g., nab-paclitaxel), docetaxel, topotecan, capecitabine, lecovorin, temozolomide, interferon-alpha, or liposomal doxorubicin (e.g., pegylated liposomal doxorubicin).

In one preferred embodiment, the chemotherapy regimen includes the administration of carboplatin and paclitaxel; carboplatin and gemcitabine; or paclitaxel, topotecan, or pegylated liposomal doxorubicin. In a second preferred embodiment, the chemotherapy regimen includes the administration of capecitabine and paclitaxel; or capecitabine and docetaxel. In a third preferred embodiment, the chemotherapy regimen includes the administration of temozolomide and optionally radiotherapy. In a fourth preferred embodiment, the chemotherapy regimen includes the administration of fluropyrimidine, irinotecan, cisplatin, fluropyramidine and oxaliplatin; fluropyrimidine and irinotecan; fluropyramidine, lecovorin, and oxaliplatin; or ironotecan, fluoropyrimidine, and leucovorin. In a fifth preferred embodiment, the chemotherapy regimen includes the administration of paclitaxel and topotecan; or paclitaxel and cisplatin. In a sixth preferred embodiment, the chemotherapy regimen includes the administration of interferon-alpha2a.

In some embodiments, the one or more stroma signature gene is selected from the group consisting of POSTN, LOX, TIMP3, FAP, BGN, FGF1, FN1, ANGPTL2, ACTA2, MMP11, RBP4, CD36, PLVAP, PECAM1, GZMK, CD247, ABCC9, PCOLCE, CD1C, MS4A1, CD44, PMEPA1, IL7R, FBLN1, TWIST1, ID1, RAC2, GFRA1, CCR7, MAN1A1, EVI2A, PTPRC CD45RA, FCRLS, NNMT, CD27, SLA, TDO2, NUAK1, and COL4A1. In preferred embodiments, the stroma signature gene is POSTN. In other preferred embodiments, the one or more stroma signature gene(s) is POSTN and FAP; POSTN and TIMP3; POSTN and LOX; POSTN, FAP, and TIMP3; POSTN, FAP, and LOX; POSTN, TIMP3, and LOX; or POSTN, FAP, TIMP3, and LOX.

In a fourth aspect, the present invention features a method of treating a patient with cancer, the method including administering to the patient a therapeutically effective amount of a stroma-targeted agent, wherein the patient's cancer has been determined to express one or more stroma signature gene(s) at a level more than the median level for expression of the one or more stroma signature gene(s) in the cancer type.

In preferred embodiments of the above methods, the stroma-targeted agent is an anti-periostin (POSTN) antibody. In certain embodiments of the above methods, the cancer is primary, advanced, refractory, or recurrent. In other embodiments, the cancer is a gynecologic cancer selected from the group consisting of ovarian cancer, peritoneal cancer, fallopian tube cancer, cervical cancer, endometrial cancer, vaginal cancer, and vulvar cancer. In preferred embodiments, the gynecologic cancer is ovarian cancer. In yet other embodiments of the above methods, the cancer is selected from the group consisting of colorectal cancer, breast cancer, non-small cell lung cancer (NSCLC), kidney cancer (renal cell carcinoma), or brain cancer (glioblastoma).

In a fifth aspect, the invention provides methods of determining the stage of ovarian cancer in a patient. The methods include determining the expression level of POSTN in a sample (e.g., a tumor tissue sample, a blood sample, or a serum sample) obtained from the patient. Detection of an increased level of expression of POSTN in the patient sample, relative to a control, indicates an advanced stage of ovarian cancer (e.g., FIGO ovarian cancer stage III or IV). In certain embodiments, the control is the median level of POSTN expression in a population of patients having ovarian cancer, while in other embodiments, the control is the median level of POSTN expression in a population of patients having FIGO stage I and/or FIGO stage II ovarian cancer. Optionally, the methods also include a step of administering a therapy to the patient, if the patient is determined to have ovarian cancer that is in an advanced stage.

Other features and advantages of the invention will be apparent from the detailed description, the drawings, and the claims.

### BRIEF DESCRIPTION OF THE DRAWINGS

The application file contains at least one drawing executed in color. Copies of this patent or patent application with color drawings will be provided by the Office upon request and payment of the necessary fee.

FIGS. 1A-1D show the identification of a “reactive stroma” gene signature up-regulated in primary chemotherapy-resistant ovarian tumors. (A) Hierarchical clustering of the top 14 most differentially expressed genes (false discovery rate (FDR) ≦10%, fold change ≧1.5) between 32 Plat-R primary and 26 Plat-S primary ovarian tumors. Clinically defined response to primary chemotherapy, TP53 mutation status, and 7 recurrently amplified genes (≧4 copies) are annotated at the bottom; (B) Hierarchical clustering of the top 65 most differentially expressed genes (FDR ≦10%, fold change ≧1.5) between 27 patient-matched Plat-R primary and Plat-R recurrent ovarian tumors; (C) Venn diagram of common signature genes significantly differentially expressed in Plat-R primary and recurrent tumors; (D) Gene expression of the four reactive stroma signature genes in 26 Plat-S primary, 32 Plat-R primary, and 27 Plat-R recurrent tumors.

FIG. 2 is a series of plots showing mRNA expression levels of the four reactive stroma signature genes that are highly correlated with one another.

FIGS. 3A-3B show in situ analysis of the reactive stroma signature genes POSTN, LOX, and FAP by RNA ISH and IHC. (A) Representative ISH and IHC images from a Plat-S primary tumor, a patient-matched Plat-R primary tumor prior to chemotherapy, and recurrent tumors post chemotherapy at disease progression. Images in the left two columns: 2-plex chromogenic RNA ISH for detection of POSTN, LOX, and singleplex RNA ISH for detection of FAP mRNA localization. Images in the right three columns: IHC staining for POSTN, FAP, and aSMA protein localization. Bar=100 um. (B) Summary of ISH scores and IHC scores in all 85 samples (POSTN and FAP ISH) or five representative tumor specimens (LOX ISH, POSTN, and FAP IHC) from each of the response group: Plat-S primary, patient-matched Plat-R primary, and recurrent tumors. Both ISH H-score (Material and Methods, plotted with means and standard deviations) and IHC overall score were determined in tumor and stromal cells respectively. *p<0.05, **p<0.01.

FIGS. 4A-4C show that POSTN expression levels are correlated with the desmoplasia phenotype in vivo, and that POSTN promotes chemotherapy-resistance in EOC cells in vitro. (A) Increased desmoplasia is correlated with POSTN expression and primary chemotherapy-resistance. Representative high magnification images of hematoxylin and eosin (H&E) staining of tumor specimens (upper panels) and POSTN ISH images (lower panel) are shown. Desmoplasia scores were defined as follows: 0=no desmoplasia, 1=few scattered desmoplastic foci abutting cancer cells, 2=several desmoplastic foci abutting cancer cells or moderate confluent (wider) desmoplasia, but not present throughout the section, 3=desmoplastic reaction throughout section, associated with most cancer cells. Labels: DS=desmoplastic stroma, NS=normal stroma, TC =tumor cells. Arrows point to examples of tumor cells. A dotted line encircles a region containing tumor cells. Size bars, 100 μm. (B) Summary of desmoplasia scores in 21 Plat-S primary, 18 Plat-R primary, and 21 Plat-R recurrent tumor specimens; (C) POSTN promotes chemotherapy-resistance in chemotherapy-sensitive ES2 ovarian cells in vitro. 96-well plates were coated with recombinant protein FN1 or POSTN or left uncoated before cells were plated into each well. 10 μM carboplatin or 10 nM taxol was then added to each well on the next day. Cell-Titre Glo® reagents were added at 72 hours after compound treatment to measure cell viability. The viability in coated wells was then compared with viability in uncoated wells to calculate % growth benefit.

FIGS. 5A-5B show that expression of reactive stroma genes predicts clinical outcome of front-line chemotherapy in the ICON7 study chemotherapy treatment arm. (A) Correlation of fold changes (Plat-R vs. Plat-S) between the discovery dataset (x-axis) and the independent validation set (ICON7 control arm) (y-axis). The five genes on the plot are significantly differentially expressed in both datasets (p≦0.01 and fold change ≧1.5); (B) Association of expression of reactive stroma signature genes (median cutoff) with patient outcome (PFS) from primary chemotherapy in an independent dataset (ICON7 chemotherapy treatment arm).

FIG. 6 is a series of plots showing the correlation between POSTN and known prognostic factors in ovarian cancer.

FIG. 7 shows multivariate analysis of the four stroma signature genes. Expression of five genes (POSTN, PGR, FAP, LOX, and TIMP3) dichotomized using median cutoff were analyzed using a multivariate Cox regression model to assess the strength of association for each gene. Only expression of POSTN was significant in this multivariate analysis. In addition, when expression of the four genes was averaged for each patient, the resulting overall stroma score did not improve association with PFS (HR=2.0, 95% CI: 1.3-3.1, p=0.0013).

FIG. 8 provides schematic diagrams of top activated networks and upstream regulator identified by pathway analysis using gene signatures associated with primary chemotherapy-resistance (Ingenuity). Down-regulated genes in chemotherapy-resistant tumors are FGFR4, CXCL10, IDO1, MMP10, and MMPI. The remaining genes vary in degree of up-regulation in chemotherapy-resistant tumors.

FIG. 9 is a plot showing that POSTN expression is highly correlated with pro-angiogenesis markers (PLVAP, PECAM1, and ANGPTL2) and M2-like macrophage markers (CD68, CD163, and CD36).

FIG. 10 is a grouped dot plot showing the range of POSTN expression in vendor procured panels of serum samples from 102 age-matched normal healthy subjects (NHS), 100 epithelial ovarian cancer (EOC) patients of unknown chemosensitivity (ovarian cancer), 43 EOC patients that are known to be platinum-resistant (Plat-R ovarian cancer), 96 lung cancer (NSCLC) patients, and 29 pancreatic cancer patients.

FIG. 11 is a grouped dot plot showing the correlation between circulating POSTN and the stage of disease in vendor procured serum samples from stage I (25) and II (6) patients (31 combined) and 69 samples from stage III patients.

### DETAILED DESCRIPTION OF THE INVENTION

### I. Introduction

The present invention provides a reactive stroma gene signature that is specifically associated with primary chemotherapy-resistance in ovarian cancer and is further up-regulated in recurrent tumors. In situ analysis of several key components of this signature, including periostin (POSTN), fibroblast activating protein (FAP), and lysyl oxidase (LOX), revealed that these genes are specifically up-regulated in tumor-associated fibroblasts in chemotherapy-resistant tumors. The reactive stroma gene signature was validated in an independent dataset from the chemotherapy treatment arm of a phase III trial, and it was shown in this validation analysis that high POSTN expression levels are associated with worse outcome (i.e., progression free survival (PFS)) for patients receiving front-line chemotherapy (carboplatin and paclitaxel).

Accordingly, the invention provides methods for identifying patients with cancer (e.g., gynecologic cancer (e.g., ovarian, peritoneal, fallopian tube, cervical, endometrial, vaginal, or vulvar cancer)) that is chemotherapy-resistant by determining the expression level of one or more stroma signature genes, and comparing this level of expression to the median level of expression of the one or more stroma signature genes in the cancer type. Detection of expression of the one or more stroma signature genes at a level more than the median level of expression of the one or more stroma signature genes in the cancer type indicates that a patient has chemotherapy-resistant cancer. The invention also provides methods for treating patients with cancer (e.g., chemotherapy-resistant cancer) by administering a stroma-targeted or other agent to the patients. The invention further provides methods of identifying patients with cancer (e.g., chemotherapy-resistant cancer) that may benefit from administration of an anti-angiogenic agent (e.g., a VEGF antagonist, such as an anti-VEGF antibody, e.g., bevacizumab) or an immunomodulatory agent in combination with a chemotherapy regimen and/or a stroma-targeted agent.

### II. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

The terms “administration” or “administering” as used herein mean the administration of a chemotherapeutic agent (e.g., any chemotherapeutic agent described herein, see below), a stroma-targeted agent (e.g., an anti-POSTN antibody), an immunomodulatory agent, and/or an anti-angiogenic agent (e.g., an anti-VEGF antibody, such as bevacizumab), and/or a pharmaceutical composition/treatment regimen comprising a chemotherapeutic agent (e.g., any described herein, see below), a stroma-targeted agent (e.g., an anti-POSTN antibody), an immunomodulatory agent, or an anti-angiogenic agent (e.g., an anti-VEGF antibody, such as bevacizumab), to a patient in need of such treatment or medical intervention by any suitable means known in the art for administration of a therapeutic antibody. Nonlimiting routes of administration include by oral, intravenous, intraperitoneal, subcutaneous, intramuscular, topical, intradermal, intranasal or intrabronchial administration (for example as effected by inhalation). Particularly preferred in context of this invention is parenteral administration, e.g., intravenous administration. With respect to bevacizumab for the treatment of colorectal cancer, the preferred dosages according to the EMEA are 5 mg/kg or 10 mg/kg of body weight given once every 2 weeks or 7.5 mg/kg or 15 mg/kg of body weight given once every 3 weeks. For the treatment of NSCLC, the preferred dosage is 15 mg/kg given once every 3 weeks by infusion in combination with carboplatin and paclitaxel. For the treatment of renal cell carcinoma, the preferred dosage is 10 mg/kg given once every 2 weeks by infusion with interferon a-2a or as a monotherapy. For the treatment of cervical cancer, the preferred dosage is 15 mg/kg given once every three weeks by infusion and administered in combination with one of the following chemotherapy regimens: paclitaxel and cisplatin or paclitaxel and topotecan. For the treatment of glioblastoma, the preferred dosage is 10 mg/kg given once every two weeks by infusion.

Methods for identifying agonists or antagonists of a polypeptide may comprise contacting a polypeptide with a candidate agonist or antagonist molecule and measuring a detectable change in one or more biological activities normally associated with the polypeptide.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 10-8 M or less, e.g. from 10-8 M to 10-13 M, e.g., from 10-9 M to 10-13 M). In certain embodiments, an anti-target antibody binds to an epitope of a target that is conserved among different species.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

The term “biological sample” or “sample” as used herein includes, but is not limited to, blood, serum, plasma, sputum, tissue biopsies, tumor tissue, and nasal samples including nasal swabs or nasal polyps.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

An “advanced” cancer is one which has spread outside the site or organ of origin, either by local invasion or metastasis.

A “refractory” cancer is one which progresses even though an anti-tumor agent, such as a chemotherapeutic agent, is being administered to the cancer patient. An example of a refractory cancer is one which is platinum refractory.

A “recurrent” cancer is one which has regrown, either at the initial site or at a distant site, after a response to initial therapy.

By “platinum-resistant” cancer is meant cancer in a patient that has progressed while the patient was receiving platinum-based chemotherapy or cancer in a patient that has progressed within, e.g., 12 months (for instance, within 6 months) after the completion of platinum-based chemotherapy. Such cancer can be said to have or exhibit “platinum-resistance.”

By “chemotherapy-resistant” cancer is meant cancer in a patient that has progressed while the patient is receiving a chemotherapy regimen or cancer in a patient that has progressed within, e.g., 12 months (for instance, within 6 months) after the completion of a chemotherapy regimen. Such cancer can be said to have or exhibit “chemotherapy-resistance.”

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

A “chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, Astra7eneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; Astra7eneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ, antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943, 533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP 659,439 A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, Astra7eneca); ZM 105180 ((6-amino-4-(3-methylphenyl-amino)-quinazoline, Zeneca); BIB X-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (Astra7eneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

By “platinum-based chemotherapeutic agent” or “platin” is meant an antineoplastic drug that is a coordination complex of platinum. Examples of platinum-based chemotherapeutic agents include carboplatin, cisplatin, satraplatin, picoplatin, nedaplatin, triplatin, lipoplatin, and oxaliplatinum.

By “platinum-based chemotherapy” is meant therapy with one or more platinum-based chemotherapeutic agent, optionally in combination with one or more other chemotherapeutic agents.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

A sample, cell, tumor, or cancer which “has been determined to express” or “expresses” a stroma signature gene at a level more than the median expression level for the stroma signature gene in a type of cancer (or in a cancer type, wherein the “cancer type” is meant to include cancerous cells (e.g., tumor cells, tumor tissues) as well as non-cancerous cells (e.g., stromal cells, stromal tissues) that surround the cancerous/tumor environment) is one in which the expression level of a stroma signature gene is considered to be a “high stroma signature gene expression level” to a skilled person for that type of cancer. Generally, such a level will be in the range from about 50% up to about 100% or more (e.g., 50%, 55%, 60%, 65%, 70%, 75%, 80%, 85%, 90%, 95%, 100%, or more)) relative to stroma signature gene levels in a population of samples, cells, tumors, or cancers of the same cancer type. For instance the population that is used to arrive at the median expression level may be ovarian cancer samples generally, or subgroupings thereof, such as chemotherapy-resistant ovarian cancer, platinum-resistant ovarian cancer, as well as advanced, refractory, or recurrent ovarian cancer samples.

By “cancer is or has been determined to express” or “cancer expresses,” used in reference to a particular biomarker (e.g., one or more stroma signature genes, e.g., POSTN), means expression of the biomarker(s) (e.g., one or more stroma signature genes, e.g., POSTN) in a cancer-associated biological environment (e.g., expression of the biomarker(s) in the tumor cells), tumor-associated cells (e.g., tumor--associated stromal cells, such as tumor-associated fibroblasts), as determined using a diagnostic test, any of the detection methods described herein, or the similar. For example, expression of POSTN can be determined using the total periostin or total POSTN assay. The term “total POSTN assay” refers to an assay that measures the levels of total POSTN in a biological sample. In one embodiment, the total POSTN levels are measured using anti-POSTN antibodies. In another embodiment, the anti-POSTN antibodies are the anti-POSTN antibodies described herein. In another example, the total POSTN levels are measured using one or more nucleic acid sequences antisense to mRNA encoding POSTN isoforms 1-4. In some embodiments, the total POSTN assay comprises the use of (1) an antibody comprising the sequences SEQ ID NO: 1 and SEQ ID NO:2 (the “25D4” antibody) and/or an antibody comprising the sequences of SEQ ID NO:3 and SEQ ID NO:4 (the “23B9” antibody) to bind POSTN in a biological sample, (2) an antibody comprising the variable region sequences SEQ ID NO: 1 and SEQ ID NO:2 and/or an antibody comprising the variable region sequences of SEQ ID NO:3 and SEQ ID NO:4 to bind POSTN in a biological sample, (3) an antibody comprising the HVR sequences of SEQ ID NO: 1 and SEQ ID NO:2 and/or an antibody comprising the HVR sequences of SEQ ID NO:3 and SEQ ID NO:4 to bind POSTN in a biological sample, (4) an antibody comprising the HVR sequences that are 95% or more identical to the HVR sequences of SEQ ID NO: 1 and SEQ ID NO:2 and/or an antibody comprising HVR sequences that are 95% or more identical to the HVR sequences of SEQ ID NO: 3 and SEQ ID NO:4.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md. 1991.

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al, Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al, supra. In one embodiment, for the VH, the subgroup is subgroup III as in Kabat et al, supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (HI, H2, H3), and three in the VL (LI, L2, L3). HVRs generally comprise amino acid residues from the hypervariable loops and/or from the “complementarity determining regions” (CDRs), the latter typically being of highest sequence variability and/or involved in antigen recognition. An HVR region as used herein comprise any number of residues located within positions 24-36 (for HVRL1), 46-56 (for HVRL2), 89-97 (for HVRL3), 26-35B (for HVRH1), 47-65 (for HVRH2), and 93-102 (for HVRH3).

An “immunoconjugate” is an antibody conjugated to one or more heterologous molecule(s), including but not limited to a cytotoxic agent.

The term “immunomodulatory agent” refers to an agent that induces, enhances, or suppresses an immune response. Immunomodulatory agents designed to elicit or amplify an immune response are activation immunomodulatory agents. Immunomodulatory agents designed to reduce or suppress an immune response are suppression immunomodulatory agents. For example, suppression immunomodulatory agents can be TDO2, CD36, GZMK, CD247, CD1C, CSF1R, IDOL IL7R, or CCR7 antagonists. The term “antagonist” is used in the broadest sense, and includes any molecule that partially or fully blocks, inhibits, or neutralizes a biological activity of a native polypeptide. Such agents (e.g., antagonists) include polypeptide(s) (e.g., an antibody, such as an anti-CSF1R antibody (RG7155), an immunoadhesin or a peptibody), an aptamer or a small molecule that can bind to a protein or a nucleic acid molecule that can bind to a nucleic acid molecule encoding a target identified herein (i.e., siRNA) that directly or indirectly target cells of the immune system (e.g., T effector cells, T regulatory cells, B cells, NK cells, inflammatory cells, antigen presenting cells (e.g., dendritic cells, macrophage), etc.). In some embodiments, immunomodulatory agents can specifically bind to receptors on cells of the immune system to affect the activity of the immune cells. In other embodiments, immunomodulatory agents target genes involved in immune signaling pathways and/or modulate activity of immune cells.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-target antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose or “extended” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used according to the methods provided herein may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CHI, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The phrase “a patient suffering from” in accordance with the invention refers to a patient showing clinical signs of cancer (e.g., a gynecologic cancer (e.g., ovarian, peritoneal, fallopian tube, cervical, endometrial, vaginal, or vulvar cancer) or breast cancer (e.g., metastatic MBC; also see below)). The phrase “being susceptible to” or “being prone to,” in the context of cancer, refers to an indication disease in a patient based on, e.g., a possible genetic predisposition, a pre- or eventual exposure to hazardous and/or carcinogenic compounds, or exposure to carcinogenic physical hazards, such as radiation.

“Patient response” or “response” (and grammatical variations thereof) can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e. reduction, slowing down or complete stopping) of disease spread; (6) decrease of auto-immune response, which may, but does not have to, result in the regression or ablation of the disease lesion; (7) relief, to some extent, of one or more symptoms associated with the disorder; (8) increase in the length of disease-free presentation following treatment; and/or (9) decreased mortality at a given point of time following treatment.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

The term “small molecule” refers to an organic molecule having a molecular weight between 50 Daltons to 2500 Daltons.

The terms “stroma signature gene,” “stroma gene signature,” and “stroma signature” refer to one of the genes set forth in Tables 1-4, combinations of the genes set forth in Tables 1-4, or sub-combinations of these genes, the gene expression pattern of which correlates with cancer chemotherapy resistance. Each individual gene of a stroma signature is a “stroma signature gene.” These genes include: POSTN, LOX, BGN, FGF1, TIMP3, FN1, FAP, ANGPTL2, ACTA2, MMP11, RBP4, CD36, PLVAP, PECAM1, GZMK, CD247, ABCC9, PCOLCE, CD1C, MS4A1, CD44, PMEPA1, IL7R, FBLN1, TWIST1, ID1, RAC2, GFRA1, CCR7, MAN1A1, EVI2A, PTPRC CD45RA, FCRLS, NNMT, CD27, SLA, ESR2, KLK7, KLK6, MUC1, DTX4, FGFR4, TSPAN8, ESR1, KRT18, FUT2, HOXD10, EXO1, INADL, IGFBP2, MYCN, ERBB3, TMEM45B, PROM1, NCAM1, MKI67, CDH3, LY6E, TJP3, SLC7A11, BNIP3, PRAME, ESM1, VTCN1, CCL28, TDO2, NUAK1, COL4A1, ABCB9, RB1, ANXA1, FOXO1, PGR, and ALPP.

By “stroma-targeted agent” is meant an agent that targets directly or indirectly the components of the tumor stroma (e.g., fibroblasts, endothelia cells, pericytes, leukocytes, extracellular matrix, etc.). A stroma-targeted agent can directly or indirectly affect the activity of any one of the genes of the stroma signature gene set forth herein by, e.g., binding to or otherwise affecting the activity of the target gene or a protein it encodes. A stroma-targeted agent can also target the tumor stroma in a different manner without affecting the activity of any one of the genes of the stroma signature (or a corresponding polypeptide) as set forth herein. Such agents can include, e.g., small molecules, aptamers, polypeptides (which include, e.g., immunoadhesins, antibodies, peptibodies, and peptides), and RNA therapeutics (which include, e.g., small interfering RNA (siRNA), microRNA (miRNA), anti-sense oligonucleotides, and steric-blocking oligonucleotides).

“Survival” refers to the patient remaining alive, and includes overall survival as well as progression free survival.

“Overall survival” refers to the patient remaining alive for a defined period of time, such as 1 year, 5 years, etc. from the time of diagnosis or treatment.

The phrase “progression-free survival” in the context of the present invention refers to the length of time during and after treatment during which, according to the assessment of the treating physician or investigator, a patient's disease does not become worse, i.e., does not progress. As the skilled person will appreciate, a patient's progression-free survival is improved or enhanced if the patient experiences a longer length of time during which the disease does not progress as compared to the average or mean progression free survival time of a control group of similarly situated patients.

By “extending survival” is meant increasing overall or progression free survival in a treated patient relative to an untreated patient (i.e., relative to a patient not treated with a stroma-targeted agent (e.g., an anti-POSTN antibody), an immunomodulatory agent, an anti-angiogenic agent (e.g., a VEGF antagonist, e.g., an anti-VEGF antibody, such as bevacizumab), or relative to a patient who does not express a stroma signature gene at the designated level, and/or relative to a patient treated with a chemotherapeutic agent (e.g., any described herein) who is chemotherapy-sensitive.

By “standard of care” herein is intended the anti-tumor agent or agents that are routinely used to treat a particular form of cancer. For example, for platinum-resistant ovarian cancer, a standard of care is a combination of carboplatin and paclitaxel.

The terms “therapeutically effective amount” or “effective amount” refer to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), improve survival (including overall survival and progression free survival) and/or improve one or more symptoms of cancer (e.g. as assessed by FOSI). Most preferably, the therapeutically effective amount of the drug is effective to improve progression free survival (PFS) and/or overall survival (OS).

The term “total periostin (POSTN)” as used herein refers to at least isoforms 1, 2, 3 and 4 of periostin. Human POSTN isoforms 1, 2, 3 and 4 are known in the art as comprising the following amino acid sequences: NP 006466.2; NP 001129406.1, NP 001129407.1, and NP 001129408.1, respectively, according to the NCBI database (SEQ ID NOs: 19-22 of US 2012/0156194, respectively, which is incorporated herein by reference in connection with these sequences and SEQ ID NO:23). An additional form of POSTN is described in US 2012/0156194. This isoform is referred to herein as “isoform 5” and has been partially sequenced. Isoform 5 comprises the amino acid sequence of SEQ ID NO:23 of US 2012/0156194. In one embodiment, the isoforms of POSTN are human POSTNs. In a further embodiment, the term total POSTN includes isoform 5 of human POSTN in addition to isoforms 1-4. In another embodiment, total POSTN is total serum POSTN or total plasma POSTN (i.e., total POSTN from a serum sample obtained from whole blood or a plasma sample obtained from whole blood, respectively, the whole blood obtained from a patient).

The term “periostin (POSTN) antibody” or “anti-POSTN antibody” refers to an antibody that binds to an isoform of POSTN. In one embodiment, the POSTN is human POSTN. In one embodiment, the antibody comprises the sequences SEQ ID NO: 1 and SEQ ID NO:2 (the “25D4” antibody) or comprises the sequences of SEQ ID NO:3 and SEQ ID NO:4 (the “23B9” antibody). In another embodiment, the antibody comprises the variable region sequences of SEQ ID NO: 1 and SEQ ID NO:2 or comprises the variable region sequences of SEQ ID NO:3 and SEQ ID NO:4. In another embodiment, the antibody comprising the HVR sequences of SEQ ID NO: 1 and SEQ ID NO:2 or the HVR sequences of SEQ ID NO:3 and SEQ ID NO:4. In another embodiment, the antibody comprises the HVR sequences that are 95% or more identical to the HVR sequences of SEQ ID NO: 1 and SEQ ID NO:2 and/or an antibody comprising HVR sequences that are 95% or more identical to the HVR sequences of SEQ ID NO:3 and SEQ ID NO:4.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, methods and compositions of the invention are useful in attempts to delay development of a disease or disorder.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al, J. Immunol. 150:880-887 (1993); Clarkson et al, Nature 352:624-628 (1991).

A “VEGF antagonist” or “VEGF-specific antagonist” refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors, VEGF signaling, and VEGF mediated angiogenesis and endothelial cell survival or proliferation. For example, a molecule capable of neutralizing, blocking, inhibiting, abrogating, reducing, or interfering with VEGF biological activities can exert its effects by binding to one or more VEGF receptor (VEGFR) (e.g., VEGFR1, VEGFR2, VEGFR3, membrane-bound VEGF receptor (mbVEGFR), or soluble VEGF receptor (sVEGFR)). Included as VEGF-specific antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF₁₂₁-gelonin (Peregrine). VEGF-specific antagonists also include antagonist variants of VEGF polypeptides, antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. VEGF antagonists also include polypeptides that bind to VEGFR, anti-VEGFR antibodies, and antigen-binding fragments thereof, and derivatives which bind to VEGFR thereby blocking, inhibiting, abrogating, reducing, or interfering with VEGF biological activities (e.g., VEGF signaling), or fusions proteins. VEGF-specific antagonists also include nonpeptide small molecules that bind to VEGF or VEGFR and are capable of blocking, inhibiting, abrogating, reducing, or interfering with VEGF biological activities. Thus, the term “VEGF activities” specifically includes VEGF mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF. In some embodiments, the VEGF inhibited by the VEGF-specific antagonist is VEGF (8-109), VEGF (1-109), or VEGF₁₆₅.

As used herein VEGF antagonists can include, but are not limited to, anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGFA antibodies (e.g., bevacizumab, sevacizumab), anti-VEGFB antibodies, anti-VEGFC antibodies (e.g., VGX-100), anti-VEGFD antibodies, and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib).

An “anti-VEGF antibody” is an antibody that binds to VEGF with sufficient affinity and specificity. In certain embodiments, the antibody will have a sufficiently high binding affinity for VEGF, for example, the antibody may bind hVEGF with a Kd value of between 100 nM-1 pM. Antibody affinities may be determined, e.g., by a surface plasmon resonance based assay (such as the BIAcore assay as described in PCT Application Publication No. WO2005/012359); enzyme-linked immunoabsorbent assay (ELISA); and competition assays (e.g. RIA's).

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as P1GF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (1997) Cancer Res. 57:4593-4599, including but not limited to the antibody known as bevacizumab (BV; AVASTIN®).

The anti-VEGF antibody “Bevacizumab (BV),” also known as “rhuMAb VEGF” or “AVASTIN®,” is a recombinant humanized anti-VEGF monoclonal antibody generated according to Presta et al. (1997) Cancer Res. 57:4593-4599. It comprises mutated human IgG1 framework regions and antigen-binding complementarity-determining regions from the murine anti-hVEGF monoclonal antibody A.4.6.1 that blocks binding of human VEGF to its receptors. Approximately 93% of the amino acid sequence of bevacizumab, including most of the framework regions, is derived from human IgG1, and about 7% of the sequence is derived from the murine antibody A4.6.1. Bevacizumab has a molecular mass of about 149,000 daltons and is glycosylated. Bevacizumab and other humanized anti-VEGF antibodies are further described in U.S. Pat. No. 6,884,879 issued Feb. 26, 2005, the entire disclosure of which is expressly incorporated herein by reference. Additional preferred antibodies include the G6 or B20 series antibodies (e.g., G6-31, B20-4.1), as described in PCT Application Publication No. WO 2005/012359. For additional preferred antibodies see U.S. Pat. Nos. 7,060,269, 6,582,959, 6,703,020; 6,054,297; W098/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89.

### III. Methods of Prognosis, Diagnosis, and Detection

The present invention relates to the identification, selection, and use of biomarkers of cancer (e.g., a gynecologic cancer (e.g., ovarian, peritoneal, fallopian tube, cervical, endometrial, vaginal, or vulvar cancer)) that are associated with resistance to chemotherapeutic agents (e.g., platinum-based chemotherapeutic agents, e.g., cisplatin, carboplatin, oxaliplatin, straplatin, picoplatin, dedaplatin, triplatin, lipoplatin, etc.). In this respect, the invention relates to the use of tumor stromal component (e.g., tumor-associated fibroblast) expression profile(s) in patients with cancer (e.g., a gynecologic cancer (e.g., ovarian, peritoneal, fallopian tube, cervical, endometrial, vaginal, or vulvar cancer)) who have been determined to have chemotherapy-resistant cancer or chemotherapy-sensitive cancer, to identify biomarkers associated with resistance to chemotherapy agents (e.g., platinum-based chemotherapeutic agents, such as cisplatin, carboplatin, oxaliplatin, straplatin, picoplatin, dedaplatin, triplatin, lipoplatin, etc.). The biomarkers of the invention are listed herein, e.g., in Tables 1-4.

The invention provides methods for identifying patients with cancer (e.g., a gynecologic cancer (e.g., ovarian, peritoneal, fallopian tube, cervical, endometrial, vaginal, or vulvar cancer)) that is chemotherapy-resistant by determining the expression level of one or more stroma signature genes (e.g., one or more of the genes listed in Tables 1-4 and/or combinations thereof), and comparing the expression level of the stroma signature gene to the median level for expression of the stroma signature gene in the cancer type. In some embodiments, the patient is determined to have cancer that is chemotherapy-resistant if expression of the stroma signature gene (e.g., any of the genes in Tables 1 and 3 and/or combinations thereof) is at a level more than the median level for expression of the stroma signature gene in the cancer type. In other embodiments, the patient is determined to have cancer that is chemotherapy-resistant if expression of the stroma signature gene (e.g., any of the genes in Tables 2 and 4 and/or combinations thereof) is at a level less than the median level for expression of the stroma signature gene in the cancer type. The invention also provides methods of identifying patients with cancer (e.g., gynecologic cancer (e.g., ovarian, peritoneal, fallopian tube, cervical, endometrial, vaginal, or vulvar cancer)) that is chemotherapy-sensitive by determining the expression level of a stroma signature gene (e.g., one or more of the genes listed in Tables 1-4 and/or combinations thereof) and comparing the expression level of the stroma signature gene to the median level for expression of the stroma signature gene in the cancer type. In some embodiments, the patient is determined to have cancer that is chemotherapy-sensitive if expression of the stroma signature gene (e.g., any of the genes in Tables 1 and 3 and/or combinations thereof) is at a level that is less than the median level for expression of the stroma signature gene in the cancer type. In other embodiments, the patient is determined to have cancer that is chemotherapy-sensitive if expression of the stroma signature gene (e.g., any of the genes in Tables 2 and 4 and/or combinations thereof) is at a level more than the median level for expression of the stroma signature gene in the cancer type. Optionally, these methods are carried out prior to administering a chemotherapeutic agent in order to provide the patient with a pre-administration diagnosis of chemotherapy resistance.

The invention also provides methods of prognosis as to the likelihood of benefiting from chemotherapy with particular chemotherapeutic agents (e.g., carboplatin, cisplatin, oxaliplatin, or any agents described herein, see above) and/or the likelihood of benefiting from alternative anti-cancer therapy in addition to or instead of chemotherapy (e.g., administering anti-angiogenesis agents, immunomodulatory agents, and/or stroma-targeting agents (e.g., an anti-POSTN antibody)). These methods involve determining the expression level of a stroma signature gene (e.g., one or more of the genes listed in Tables 1-4 and/or combinations thereof) and comparing the expression level of the stroma signature gene to the median level for expression of the stroma signature gene in the cancer type. In some embodiments, the patient is determined to likely benefit from administration of an anti-cancer therapy (e.g., anti-angiogenesis therapy, immunotherapy, stroma-targeted therapy, etc.) in addition to or instead of chemotherapy if expression of the stroma signature gene (e.g., any of the genes in Tables 1 and 3 and/or combinations thereof) is at a level more than the median level for expression of the stroma signature gene in the cancer type. In other embodiments, the patient is determined to likely benefit from administration of an anti-cancer therapy (e.g., anti-angiogenesis therapy, immunotherapy, stroma-targeted therapy, etc.) in addition to or instead of chemotherapy if expression of the stroma signature gene (e.g., any of the genes in Tables 2 and 4 and/or combinations thereof) is at a level less than the median level for expression of the stroma signature gene in the cancer type. Optionally, these methods include administering the anti-cancer therapy (e.g., administering an anti-angiogenesis agent (e.g., a VEGF antagonist, such as an anti-VEGF antibody, e.g., bevacizumab), an immunomodulatory agent, and/or a stroma-targeted agent (e.g., an anti-POSTN antibody)) to the patient in combination with a chemotherapy regimen or as a monotherapy.

TABLE 1
Differentially expressed up-regulated genes in platinum-resistant vs.
platinum-sensitive primary ovarian tumors

| | | |
|---|---|---|
| POSTN (Gene ID No.: 10631) | FAP (Gene ID | TIMP3 (Gene ID |
| | No.: 2191) | No.: 7078) |
| LOX (Gene ID No.: 4015) | TDO2 (Gene ID | NUAK1 (Gene ID |
| | No.: 6999) | No.: 9891) |
| COL4A1 (Gene ID No.: 1282) | | |

TABLE 2
Differentially expressed down-regulated genes in platinum-resistant vs.
platinum-sensitive primary ovarian tumors

| | | |
|---|---|---|
| ABCB9 (Gene ID No.: 23457) | FGFR4 (Gene ID | RB1 (Gene ID |
| | No.: 2264) | No.: 5925) |
| ANXA1 (Gene ID No.: 301) | FOXO1 (Gene ID | PGR (Gene ID |
| | No.: 2308) | No.: 5241) |
| ALPP (Gene ID No.: 250) | | |

TABLE 3
Differentially expressed up-regulated genes in platinum-resistant recurrent ovarian
tumors vs. platinum-resistant primary ovarian tumors tumors

| | | |
|---|---|---|
| LOX (Gene ID No.: 4015) | BGN (Gene ID No.: 633) | FGF1 (Gene ID No.: 2246) |
| TIMP3 (Gene ID No.: 7078) | FN1 (Gene ID No.: 2335) | FAP (Gene ID No.: 2191) |
| ANGPTL2 (Gene ID No.: | POSTN (Gene ID No.: 10631) | ACTA2 (Gene ID No.: 59) |
| 23452) | | |
| MMP11 (Gene ID No.: 4320) | RBP4 (Gene ID No.: 5950) | CD36 (Gene ID No.: 948) |
| PLVAP (Gene ID No.: 83483) | PECAM1 (Gene ID No.: | GZMK (Gene ID No.: 3003) |
| | 5175) | |
| CD247 (Gene ID No.: 919) | ABCC9 (Gene ID No.: 10060) | PCOLCE (Gene ID No.: |
| | | 5118) |
| CD1C (Gene ID No.: 911) | MS4A1 (Gene ID No.: 931) | CD44 (Gene ID No.: 960) |
| PMEPA1 (Gene ID No.: | IL7R (Gene ID No.: 3575) | FBLN1 (Gene ID No.: 2192) |
| 56937) | | |
| TWIST1 (Gene ID No.: 7291) | ID1 (Gene ID No.: 3397) | RAC2 (Gene ID No.: 5880) |
| GFRA1 (Gene ID No.: 2674) | CCR7 (Gene ID No.: 1236) | MAN1A1 (Gene ID No.: |
| | | 4121) |
| EVI2A (Gene ID No.: 2123) | PTPRC/CD45RA (Gene ID | FCRL5 (Gene ID No.: 83416) |
| | No.: 5788 | |
| NNMT (Gene ID No.: 4837) | CD27 (Gene ID No.: 939) | SLA (Gene ID No.: 6503) |

TABLE 4
Differentially expressed down-regulated genes in platinum-resistant recurrent ovarian
tumors vs. platinum-resistant primary ovarian tumors

| | | |
|---|---|---|
| ESR2 (Gene ID No.: 2100) | KLK7 (Gene ID No.: 5650) | KLK6 (Gene ID No.: 5653) |
| MUC1 (Gene ID No.: 4582) | DTX4 (Gene ID No.: 23220) | FGFR4 (Gene ID No.: 2264) |
| TSPAN8 (Gene ID No.: 7103) | ESR1 (Gene ID No.: 2099) | KRT18 (Gene ID No.: 3875) |
| FUT2 (Gene ID No.: 2524) | HOXD10 (Gene ID No.: | EXO1 (Gene ID No.: 9156) |
| | 3236) | |
| INADL (Gene ID No.: 10207) | IGFBP2 (Gene ID No.: 3485) | MYCN (Gene ID No.: 4613) |
| ERBB3 (Gene ID No.: 2065) | TMEM45B (Gene ID No.: | PROM1 (Gene ID No.: 8842) |
| | 120224) | |
| NCAM1 (Gene ID No.: 4684) | MKI67 (Gene ID No.: 4288) | CDH3 (Gene ID No.: 1001) |
| LY6E (Gene ID No.: 4061) | TJP3 (Gene ID No.: 27134) | SLC7A11 (Gene ID No.: |
| | | 23657) |
| BNIP3 (Gene ID No.: 664) | PRAME (Gene ID No.: | ESM1 (Gene ID No.: 11082) |
| | 23532) | |
| VTCN1 (Gene ID No.: 79679) | CCL28 (Gene ID No.: 56477) | |

*Gene ID Nos. were retrieved on Jul. 29, 2015 from the Nanostring Technologies webpage at store.nanostring.com/search.

The invention also provides methods of determining the stage of cancer in a patient. In these methods, the level of expression of one or more stroma signature genes as described herein is assessed, and an increase in expression of the gene(s) indicates a later stage of cancer. In one example, the level of, e.g., POSTN is assessed in a sample (e.g., a blood sample, such as a serum sample), and detection of an increased level of expression of the gene, e.g., POSTN, indicates a later (e.g., FIGO stage III (e.g., stage IIIA, IIIB, or IIIC) or IV) stage of EOC. The level of expression of the signature gene(s) in the sample can be compared to, e.g., the median expression level of the gene in a population of patients having the cancer type, in general, or can be compared to levels determined to be associated with particular stages (e.g., early stages, such as FIGO stage I or FIGO stage II EOC) of the cancer type.

The expression level of a stroma signature gene may be assessed by any method known in the art suitable for determination of specific protein levels in a patient sample, and is preferably determined by an immunohistochemical (“IHC”) method employing antibodies specific for a stroma signature gene. Such methods are well known and routinely implemented in the art, and corresponding commercial antibodies and/or kits are readily available. Preferably, the expression levels of the marker/indicator proteins of the invention are assessed using the reagents and/or protocol recommendations of the antibody or kit manufacturer. The skilled person will also be aware of further means for determining the expression level of a stroma signature gene by IHC methods. Therefore, the expression level of one or more of the markers/indicators of the invention can be routinely and reproducibly determined by a person skilled in the art without undue burden. However, to ensure accurate and reproducible results, the invention also encompasses the testing of patient samples in a specialized laboratory that can ensure the validation of testing procedures.

Preferably, the expression level of a stroma signature gene is assessed in a biological sample that contains or is suspected to contain cancer cells. The sample may be, for example, an ovarian tissue resection, an ovarian tissue biopsy, or a metastatic lesion obtained from a patient suffering from, suspected to suffer from, or diagnosed with cancer (e.g., a gynecologic cancer, in particular ovarian cancer). Preferably, the sample is a sample of ovarian tissue, a resection or biopsy of an ovarian tumor, a known or suspected metastatic ovarian cancer lesion or section, or a blood sample, e.g., a peripheral blood sample, known or suspected to comprise circulating cancer cells, e.g., ovarian cancer cells. The sample may comprise both cancer cells, i.e., tumor cells, and non-cancerous cells, and, in certain embodiments, comprises both cancerous and non-cancerous cells (e.g., preferably, the samples contain stromal cells). In aspects of the invention comprising the determination of gene expression in stroma components, the sample comprises both cancer/tumor cells and non-cancerous cells that are, e.g., associated with the cancer/tumor cells (e.g., tumor associated fibroblasts, endothelial cells, pericytes, the extra-cellular matrix, and/or various classes of leukocytes). In other aspects, the skilled artisan, e.g., a pathologist, can readily discern cancer cells from non-cancerous (e.g., stromal cells, endothelial cells, etc.). Methods of obtaining biological samples including tissue resections, biopsies, and body fluids, e.g., blood samples comprising cancer/tumor cells, are well known in the art. In some embodiments, the sample obtained from the patient is collected prior to beginning any chemotherapeutic or other treatment regimen or therapy, e.g., therapy for the treatment of cancer or the management or amelioration of a symptom thereof. Therefore, in some embodiments, the sample is collected before the administration of chemotherapeutics or other agents, or the start of a chemotherapy or other treatment regimen.

In addition to the methods described above, the invention also encompasses further immunohistochemical methods for assessing the expression level of one or more stroma signature gene, such as by Western blotting and ELISA-based detection. As is understood in the art, the expression level of the marker/indicator proteins of the invention may also be assessed at the mRNA level by any suitable method known in the art, such as Northern blotting, real time PCR, and RT PCR. Immunohistochemical- and mRNA-based detection methods and systems are well known in the art and can be deduced from standard textbooks, such as Lottspeich (Bioanalytik, Spektrum Akademisher Verlag, 1998) or Sambrook and Russell (Molecular Cloning: A Laboratory Manual, CSH Press, Cold Spring Harbor, N.Y., U.S.A., 2001). In preferred embodiments, the method for detecting mRNA levels of a stroma signature gene is performed using RNA in situ hybridization (RNA ISH) (e.g., see below). The described methods are of particular use for determining the expression levels of a stroma signature gene in a patient or group of patients relative to control levels established in a population diagnosed with advanced stages of cancer (e.g., a gynecologic cancer, such as ovarian cancer).

For use in the detection methods described herein, the skilled person has the ability to label the polypeptides or oligonucleotides encompassed by the present invention. As routinely practiced in the art, hybridization probes for use in detecting mRNA levels and/or antibodies or antibody fragments for use in IHC methods can be labeled and visualized according to standard methods known in the art. Non-limiting examples of commonly used systems include the use of radiolabels, enzyme labels, fluorescent tags, biotin-avidin complexes, chemiluminescence, and the like.

The expression level of one or more of a stroma signature gene can also be determined on the protein level by taking advantage of immunoagglutination, immunoprecipitation (e.g., immunodiffusion, immunelectrophoresis, immune fixation), western blotting techniques (e.g., in situ immuno histochemistry, in situ immuno cytochemistry, affinity chromatography, enzyme immunoassays), and the like. Amounts of purified polypeptide may also be determined by physical methods, e.g., photometry. Methods of quantifying a particular polypeptide in a mixture usually rely on specific binding, e.g., of antibodies.

As mentioned above, the expression level of the marker/indicator proteins according to the present invention may also be reflected in increased or decreased expression of the corresponding gene(s) encoding the stroma signature gene. Therefore, a quantitative assessment of the gene product prior to translation (e.g. spliced, unspliced or partially spliced mRNA) can be performed in order to evaluate the expression of the corresponding gene(s). The person skilled in the art is aware of standard methods to be used in this context or may deduce these methods from standard textbooks (e.g. Sambrook, 2001). For example, quantitative data on the respective concentration/amounts of mRNA encoding one or more of a stroma signature gene as described herein can be obtained by Northern Blot, Real Time PCR, and the like.

### IV. Methods of Treatment

The present invention provides methods of treating patients with cancer (e.g., a chemotherapy-resistant cancer, a chemotherapy-sensitive cancer, primary cancer, advanced cancer, refractory cancer, and/or recurrent cancer). The methods include administering to the patient a therapeutically effective amount of a stroma-targeted agent (e.g., an anti-POSTN antibody), if the patient's cancer has been determined to express a stroma signature gene (e.g., one or more genes described in Tables 1 and 3) at a level more than the median level for expression of the stroma signature gene in the cancer type or determined to express a stroma signature gene (e.g., one or more genes described in Tables 2 and 4) at a level less than the median level for expression of the stroma signature gene in the cancer type. In some embodiments, the stroma-targeted agent can be administered as a monotherapy. In other embodiments, the stroma-targeted agent can be administered in combination with a chemotherapy regimen, radiation therapy, and/or immunotherapy.

In particular embodiments, the stroma-targeted agent is an agent that binds to periostin (POSTN). In certain embodiments, the agent that binds to POSTN is an isolated antibody (i.e., an anti-periostin (POSTN) antibody (anti-POSTN antibody). In particular embodiments, the anti-POSTN antibody can bind to isoforms 1-4 of human POSTN with good affinity.

In one embodiment, the antibody comprises the sequences SEQ ID NO: 1 and SEQ ID NO:2 (the “25D4” antibody) or comprises the sequences of SEQ ID NO:3 and SEQ ID NO:4 (the “23B9” antibody). In another embodiment, the antibody comprises the variable region sequences SEQ ID NO: 1 and SEQ ID NO:2 or comprises the variable region sequences of SEQ ID NO:3 and SEQ ID NO:4. In another embodiment, the antibody comprising the HVR sequences of SEQ ID NO: 1 and SEQ ID NO:2 or the HVR sequences of SEQ ID NO:3 and SEQ ID NO:4. In another embodiment, the antibody comprises the HVR sequences that are 95% or more identical to the HVR sequences of SEQ ID NO: 1 and SEQ ID NO:2 and/or an antibody comprising HVR sequences that are 95% or more identical to the HVR sequences of SEQ ID NO:3 and SEQ ID NO:4.

In any of the above embodiments, an anti-POSTN antibody can be humanized. In one embodiment, an anti-POSTN antibody comprises HVRs as in any of the above embodiments, and further comprises an acceptor human framework, e.g. a human immunoglobulin framework or a human consensus framework.

In another aspect, an anti-POSTN antibody comprises a heavy chain variable domain (VH) sequence having at least 90%, 91%, 92%, 93%, 94%, 95%, 96%, 97%, 98%, 99%), or 100%) sequence identity to the amino acid sequence of SEQ ID NO: 1. In certain embodiments, a VH sequence having at least 90%, 91%, 92%, 93%, 94%, 95%, 96%, 97%, 98%o, or 99%) identity contains substitutions (e.g., conservative substitutions), insertions, or deletions relative to the reference sequence, but an anti-POSTN antibody comprising that sequence retains the ability to bind to periostin. In certain embodiments, a total of 1 to 10 amino acids have been substituted, inserted and/or deleted in SEQ ID NO: 1. In certain embodiments, substitutions, insertions, or deletions occur in regions outside the HVRs (i.e., in the FRs). Optionally, the anti-POSTN antibody comprises the VH sequence in SEQ ID NO: 1, including post-translational modifications of that sequence.

In another aspect, an anti-POSTN antibody is provided, wherein the antibody comprises a light chain variable domain (VL) having at least 90%>, 91 >, 92%, 93%>, 94%>, 95%, 96%, 97%, 98%, 99%, or 100% sequence identity to the amino acid sequence of SEQ ID NO:2. In certain embodiments, a VL sequence having at least 90%>, 91%>, 92%, 93%>, 94%, 95%, 96%, 97%, 98%, or 99% identity contains substitutions (e.g., conservative substitutions), insertions, or deletions relative to the reference sequence, but an anti-POSTN antibody comprising that sequence retains the ability to bind to periostin. In certain embodiments, a total of 1 to 10 amino acids have been substituted, inserted and/or deleted in SEQ ID NO:2. In certain embodiments, the substitutions, insertions, or deletions occur in regions outside the HVRs (i.e., in the FRs). Optionally, the anti-POSTN antibody comprises the VL sequence in SEQ ID NO:2, including post-translational modifications of that sequence.

In another aspect, an anti-POSTN antibody comprises a heavy chain variable domain (VH) sequence having at least 90%, 91%, 92%, 93%, 94%, 95%, 96%, 97%, 98%, 99%), or 100%) sequence identity to the amino acid sequence of SEQ ID NO:3. In certain embodiments, a VH sequence having at least 90%, 91%, 92%, 93%, 94%, 95%, 96%, 97%, 98%o, or 99%) identity contains substitutions (e.g., conservative substitutions), insertions, or deletions relative to the reference sequence, but an anti-POSTN antibody comprising that sequence retains the ability to bind to periostin. In certain embodiments, a total of 1 to 10 amino acids have been substituted, inserted and/or deleted in SEQ ID NO:3. In certain embodiments, substitutions, insertions, or deletions occur in regions outside the HVRs (i.e., in the FRs). Optionally, the anti-POSTN antibody comprises the VH sequence in SEQ ID NO:3, including post-translational modifications of that sequence.

In another aspect, an anti-POSTN antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:2. In certain embodiments, a VL sequence having at least 90%>, 91%>, 92%, 93%>, 94%, 95%, 96%, 97%, 98%, or 99% identity contains substitutions (e.g., conservative substitutions), insertions, or deletions relative to the reference sequence, but an anti-POSTN antibody comprising that sequence retains the ability to bind to periostin. In certain embodiments, a total of 1 to 10 amino acids have been substituted, inserted and/or deleted in SEQ ID NO:4. In certain embodiments, the substitutions, insertions, or deletions occur in regions outside the HVRs (i.e., in the FRs). Optionally, the anti-POSTN antibody comprises the VL sequence in SEQ ID NO:4, including post-translational modifications of that sequence.

In another aspect, an anti-POSTN antibody is provided, wherein the antibody comprises a VH as in any of the embodiments provided above, and a VL as in any of the embodiments provided above.

In a further aspect, the invention employs an antibody that binds to the same epitope as an anti-POSTN antibody provided herein. For example, in certain embodiments, an antibody is provided that binds to the same epitope as an anti-POSTN antibody comprising a VH sequence of SEQ ID NO: 1 and a VL sequence of SEQ ID NO:2. For example, in certain embodiments, an antibody is provided that binds to the same epitope as an anti-periostin antibody comprising a VH sequence of SEQ ID NO:3 and a VL sequence of SEQ ID NO:4.

In a further aspect of the invention, an anti-POSTN antibody according to any of the above embodiments is a monoclonal antibody, including a chimeric, humanized or human antibody. In one embodiment, an anti-POSTN antibody is an antibody fragment, e.g., a Fv, Fab, Fab′, scFv, diabody, or F(ab′)2 fragment. In another embodiment, the antibody is a full length antibody, e.g., an intact IgG1 or IgG4 antibody or other antibody class or isotype as defined herein. In another embodiment, the antibody is a bispecific antibody.

The present invention also provides methods of identifying a patient suffering from cancer who may benefit from administration of an anti-angiogenic agent (e.g., a VEGF antagonist, such as an anti-VEGF antibody, e.g., bevacizumab) or an immunomodulatory agent by determining the expression level of a stroma signature gene (e.g., any one of the genes in Tables 1-4 or combinations thereof) where the patient is administered an anti-angiogenic agent or immunomodulatory agent if expression of the stroma signature gene (e.g., any of the genes in Tables 1 and 3 and/or combinations thereof) is at a level more than the median level for expression of the stroma signature gene in the cancer type. In other embodiments, the patient is administered an anti-angiogenic agent or an immunomodulatory agent if expression of the stroma signature gene (e.g., any of the genes in Tables 2 and 4 and/or combinations thereof) is at a level less than the median level for expression of the stroma signature gene in the cancer type. The anti-angiogenic agent (e.g., a VEGF antagonist, such as an anti-VEGF antibody, e.g., bevacizumab) can be administered in combination with an immunomodulatory agent, a chemotherapy regiment, or a stroma-targeted agent (e.g., an anti-POSTN antibody).

Accordingly, the invention provides methods for treating patients with cancer (e.g., gynecologic cancer (e.g., ovarian, peritoneal, fallopian tube, cervical, endometrial, vaginal, or vulvar cancer)) that is chemotherapy-resistant, chemotherapy-sensitive, refractory, primary, advanced, or recurrent, involving administering a therapeutically effective amount of an anti-angiogenic agent (e.g., a VEGF antagonist (e.g., an anti-VEGF antibody, such as bevacizumab)) to the patient, optionally, these methods involve the co-administration of the VEGF antagonist with one or more additional chemotherapeutic agents (e.g., carboplatin and/or paclitaxel), as described further below.

Therapy with a stroma-targeted agent, immunomodulatory agent, and/or anti-angiogenic agent (e.g., a VEGF antagonist (e.g., an anti-VEGF antibody, such as bevacizumab)), optionally in combination with one or more chemotherapeutic agents (e.g., carboplatin and/or paclitaxel) preferably extends and/or improves survival, including progression free survival (PFS) and/or overall survival (OS). In one embodiment, therapy with a stroma-targeted agent, immunomodulatory agent, and/or anti-angiogenic agent (e.g., a VEGF antagonist (e.g., an anti-VEGF antibody, such as bevacizumab)) extends survival at least about 20% more than survival achieved by administering an approved anti-tumor agent, or standard of care, for the cancer being treated. In preferred embodiments, the patient has a gynecologic cancer (e.g., ovarian, peritoneal, fallopian tube, cervical, endometrial, vaginal, or vulvar cancer).

For the prevention or treatment of cancer, the dose of a stroma-targeted agent, immunomodulatory agent, and/or anti-angiogenic agent (e.g., a VEGF antagonist (e.g., an anti-VEGF antibody, such as bevacizumab)) and/or chemotherapeutic agent will depend on the type of cancer to be treated, as defined above, the severity and course of the cancer, whether the antibody is administered for preventive or therapeutic purposes, previous therapy, the patient's clinical history and response to the drug, and the discretion of the attending physician.

In one embodiment, a fixed dose of the stroma-targeted agent, immunomodulatory agent, and/or anti-angiogenic agent (e.g., a VEGF antagonist (e.g., an anti-VEGF antibody, such as bevacizumab)) is administered. The fixed dose may suitably be administered to the patient at one time or over a series of treatments. Where a fixed dose is administered, preferably it is in the range from about 20 mg to about 2000 mg. For example, the fixed dose may be approximately 420 mg, approximately 525 mg, approximately 840 mg, or approximately 1050 mg of the agent (e.g., a stroma-targeted agent, immunomodulatory agent, and/or anti-angiogenic agent (e.g., a VEGF antagonist (e.g., an anti-VEGF antibody, such as bevacizumab)). Where a series of doses are administered, these may, for example, be administered approximately every week, approximately every 2 weeks, approximately every 3 weeks, or approximately every 4 weeks, but preferably approximately every 3 weeks. The fixed doses may, for example, continue to be administered until disease progression, adverse event, or other time as determined by the physician. For example, from about two, three, or four, up to about 17 or more fixed doses may be administered.

In one embodiment, one or more loading dose(s) of the stroma-targeted agent, immunomodulatory agent, and/or anti-angiogenic agent (e.g., a VEGF antagonist (e.g., an anti-VEGF antibody, such as bevacizumab)) are administered, followed by one or more maintenance dose(s). In another embodiment, a plurality of the same dose is administered to the patient.

While the stroma-targeted agent, immunomodulatory agent, and/or anti-angiogenic agent (e.g., a VEGF antagonist (e.g., an anti-VEGF antibody, such as bevacizumab)) may be administered as a single anti-tumor agent, the patient is optionally treated with a combination of the stroma-targeted agent, immunomodulatory agent, and/or anti-angiogenic agent (e.g., a VEGF antagonist (e.g., an anti-VEGF antibody, such as bevacizumab)) and one or more (additional) chemotherapeutic agent(s). Exemplary chemotherapeutic agents herein include: gemcitabine, carboplatin, oxaliplatin, irinotecan, fluoropyrimidine (e.g., 5-FU), paclitaxel (e.g., nab-paclitaxel), docetaxel, topotecan, capecitabine, temozolomide, interferon-alpha, and/or liposomal doxorubicin (e.g., pegylated liposomal doxorubicin). In some embodiments, at least one of the chemotherapeutic agents is carboplatin or paclitaxel. The combined administration includes co-administration or concurrent administration, using separate formulations or a single pharmaceutical formulation, and consecutive administration in either order, wherein preferably there is a time period while both (or all) active agents simultaneously exert their biological activities. Thus, the chemotherapeutic agent may be administered prior to, or following, administration of the stroma-targeted agent, immunomodulatory agent, and/or anti-angiogenic agent (e.g., a VEGF antagonist (e.g., an anti-VEGF antibody, such as bevacizumab)). In this embodiment, the timing between at least one administration of the chemotherapeutic agent and at least one administration of the a stroma-targeted agent, immunomodulatory agent, and/or anti-angiogenic agent (e.g., a VEGF antagonist (e.g., an anti-VEGF antibody, such as bevacizumab)) is preferably approximately 1 month or less (3 weeks, 2, weeks, 1 week, 6 days, 5, days, 4 days, 3 days, 2 days, 1 day). Alternatively, the chemotherapeutic agent and the stroma-targeted agent, immunomodulatory agent, and/or anti-angiogenic agent (e.g., a VEGF antagonist (e.g., an anti-VEGF antibody, such as bevacizumab)) are administered concurrently to the patient, in a single formulation or separate formulations. Treatment with the combination of the chemotherapeutic agent (e.g., carboplatin and/or paclitaxel) and the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) may result in a synergistic, or greater than additive, therapeutic benefit to the patient.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g. for therapy of ovarian cancer, include: a chemotherapeutic agent such as a platinum compound (e.g., carboplatin), a taxol such as paclitaxel or docetaxel, topotecan, or liposomal doxorubicin.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of advanced stage epithelial ovarian cancer, fallopian tube cancer, or primary peritoneal cancer include: chemotherapeutic agents such as carboplatin and paclitaxel.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of platinum-sensitive epithelial ovarian cancer, fallopian tube cancer, or primary peritoneal cancer include: chemotherapeutic agents such as carboplatin and gemcitabine.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of platinum-resistant recurrent epithelial ovarian cancer, fallopian tube cancer, or primary peritoneal cancer include: a chemotherapeutic agent such as paclitaxel, topotecan, or pegylated liposomal doxorubicin.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of breast cancer, include: chemotherapeutic agents such as capecitabine, and a taxol such as paclitaxel (e.g., nab-paclitaxel) or docetaxel.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of glioblastoma, include: chemotherapeutic agents such as temozolomide, optionally in combination with radiotherapy.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of colorectal cancer, include: chemotherapeutic agents such as a fluoropyrimidine (e.g., 5-FU), paclitaxel, cisplatin, topotecan, irinotecan, fluoropyrimidine-oxaliplatin, fluoropyrimidine-irinotecan, FOLFOX4 (5-FU, lecovorin, oxaliplatin), and IFL (ironotecan, 5-FU, leucovorin).

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of renal cell carcinoma, include: chemotherapeutic agents such as interferon-alpha2a.

Particularly desired chemotherapeutic agents for combining with the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)), e.g., for therapy of cervical cancer, include: chemotherapeutic agents such as paclitaxel, cisplatin, topotecan, paclitaxel in combination with cisplatin, and paclitaxel in combination with topotecan.

A chemotherapeutic agent, if administered, is usually administered at dosages known therefore, or optionally lowered due to combined action of the drugs or negative side effects attributable to administration of the chemotherapeutic agent. Preparation and dosing schedules for such chemotherapeutic agents may be used according to manufacturers' instructions or as determined empirically by the skilled practitioner. Where the chemotherapeutic agent is paclitaxel, preferably, it is administered at a dose between about 130 mg/m² to 200 mg/m² (for example approximately 175 mg/m²), for instance, over 3 hours, once every 3 weeks. Where the chemotherapeutic agent is carboplatin, preferably it is administered by calculating the dose of carboplatin using the Calvert formula which is based on a patient's preexisting renal function or renal function and desired platelet nadir. Renal excretion is the major route of elimination for carboplatin. The use of this dosing formula, as compared to empirical dose calculation based on body surface area, allows compensation for patient variations in pretreatment renal function that might otherwise result in either underdosing (in patients with above average renal function) or overdosing (in patients with impaired renal function). The target AUC of 4-6 mg/mL/min using single agent carboplatin appears to provide the most appropriate dose range in previously treated patients.

Aside from the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) and chemotherapeutic agent, other therapeutic regimens may be combined therewith. For example, a second (third, fourth, etc.) chemotherapeutic agent(s) may be administered, wherein the second chemotherapeutic agent is an antimetabolite chemotherapeutic agent, or a chemotherapeutic agent that is not an antimetabolite. For example, the second chemotherapeutic agent may be a taxane (such as paclitaxel or docetaxel), capecitabine, or platinum-based chemotherapeutic agent (such as carboplatin, cisplatin, or oxaliplatin), anthracycline (such as doxorubicin, including, liposomal doxorubicin), topotecan, pemetrexed, vinca alkaloid (such as vinorelbine), and TLK 286. “Cocktails” of different chemotherapeutic agents may be administered.

Other therapeutic agents that may be combined with the stroma-targeted agent, immunomodulatory agent, anti-angiogenic agent (e.g., a VEGF antagonist (e.g., an anti-VEGF antibody, such as bevacizumab)), and/or chemotherapeutic agent include any one or more of: a HER inhibitor, HER dimerization inhibitor (for example, a growth inhibitory HER2 antibody such as trastuzumab, or a HER2 antibody which induces apoptosis of a HER2-overexpressing cell, such as 7C2, 7F3 or humanized variants thereof); an antibody directed against a different tumor associated antigen, such as EGFR, HERS, HE R4; anti-hormonal compound, e.g., an anti-estrogen compound such as tamoxifen, or an aromatase inhibitor; a cardioprotectant (to prevent or reduce any myocardial dysfunction associated with the therapy); a cytokine; an EGFR-targeted drug (such as TARCEVA® IRESSA® or cetuximab); a tyrosine kinase inhibitor; a COX inhibitor (for instance a COX-1 or COX-2 inhibitor); non-steroidal anti-inflammatory drug, celecoxib (CELEBREX®); farnesyl transferase inhibitor (for example, Tipifarnib/ZARNESTRA® R115777 available from Johnson and Johnson or Lonafarnib SCH66336 available from Schering-Plough); antibody that binds oncofetal protein CA 125 such as Oregovomab (MoAb B43.13); HER2 vaccine (such as HER2AutoVac vaccine from Pharmexia, or APC8024 protein vaccine from Dendreon, or HER2 peptide vaccine from GSK/Corixa); another HER targeting therapy (e.g. trastuzumab, cetuximab, ABX-EGF, EMD7200, gefitinib, erlotinib, CP724714, CI1033, GW572016, IMC-11F8, TAK165, etc); Raf and/or ras inhibitor (see, for example, WO 2003/86467); doxorubicin HC1 liposome injection (DOXIL®); topoisomerase 1 inhibitor such as topotecan; taxane; HER2 and EGFR dual tyrosine kinase inhibitor such as lapatinib/GW572016; TLK286 (TELCYTA®); EMD-7200; a medicament that treats nausea such as a serotonin antagonist, steroid, or benzodiazepine; a medicament that prevents or treats skin rash or standard acne therapies, including topical or oral antibiotic; a medicament that treats or prevents diarrhea; a body temperature-reducing medicament such as acetaminophen, diphenhydramine, or meperidine; hematopoietic growth factor, etc.

Suitable dosages for any of the above-noted co-administered agents are those presently used and may be lowered due to the combined action (synergy) of the agent and the stroma-targeted agent, immunomodulatory agent, and/or anti-angiogenic agent (e.g., a VEGF antagonist (e.g., an anti-VEGF antibody, such as bevacizumab)). In addition to the above therapeutic regimes, the patient may be subjected to surgical removal of tumors and/or cancer cells, and/or radiation therapy.

Where the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) is an antibody, preferably the administered antibody is a naked antibody. The stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) administered may be conjugated with a cytotoxic agent. Preferably, the conjugate and/or antigen to which it is bound is/are internalized by the cell, resulting in increased therapeutic efficacy of the conjugate in killing the cancer cell to which it binds. In a preferred embodiment, the cytotoxic agent targets or interferes with nucleic acid in the cancer cell. Examples of such cytotoxic agents include maytansinoids, calicheamicins, ribonucleases, and DNA endonucleases.

The stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) can be administered by gene therapy. See, for example, WO 96/07321 published Mar. 14, 1996 concerning the use of gene therapy to generate intracellular antibodies. There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus. The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:44294432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

### V. Dosages and Formulations

The stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules including but not limited to single or multiple administrations over various time-points, bolus administration, and pulse infusion are contemplated herein.

The stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a therapeutic agent of the invention (when used alone or in combination with one or more other additional therapeutic agents) will depend on the type of disease to be treated, the type of agent, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments. Depending on the type and severity of the disease, about 1 μg/kg to 15 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the agent would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). However, other dosage regimens may be useful. The progress of this therapy is easily monitored by conventional techniques and assays.

In certain embodiments, the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)) is administered as a flat dose (i.e., not weight dependent) of 37.5 mg, or a flat dose of 125 mg, or a flat dose of 250 mg. In certain embodiments, the dose is administered by subcutaneous injection once every 4 weeks for a period of time. In certain embodiments, the period of time is 6 months, one year, two years, five years, ten years, 15 years, 20 years, or the lifetime of the patient.

In another embodiment, the patient is determined to have cancer that is chemotherapy-resistant and is selected for treatment with an anti-POSTN antibody or any of the therapeutic agents as described above. In one embodiment, the cancer patient is age 18 or older. In one embodiment, the cancer patient is age 12 to 17 and the therapeutic agent is administered as a flat dose of 250 mg or a flat dose of 125 mg. In one embodiment, the cancer patient is age 6 to 11 and the therapeutic agent is administered in as a flat dose of 125 mg.

### VI. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an agent of the invention (e.g., the stroma-targeted agent, (e.g., an anti-POSTN antibody), immunomodulatory agent, and/or anti-angiogenic agent (e.g., a VEGF antagonist (e.g., an anti-VEGF antibody, such as bevacizumab)). The label or package insert indicates that the composition is used for treating the condition of choice. Moreover, the article of manufacture may comprise (a) a first container with a composition contained therein, wherein the composition comprises an agent (e.g., the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)); and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. It is understood that any of the above articles of manufacture may include an immunoconjugate of the invention in place of or in addition to the agent (e.g., the stroma-targeted agent (e.g., an anti-POSTN antibody), immunomodulatory agent, and/or anti-angiogenic agent (e.g., a VEGF antagonist (e.g., an anti-VEGF antibody, such as bevacizumab)).

### EXAMPLES

A systematic and in-depth analysis was carried out to discover, functionally characterize, and independently validate key molecular characteristics associated with chemotherapy resistance to primary treatments. For discovery, a set of patients was selected having clinically well-defined response to primary chemotherapy treatment and matched clinicopathological characteristics. For the independent validation study, tissue samples from patients enrolled in the chemotherapy control arm of a phase III clinical trial with representative intended to treat (ITT) patient population and well-balanced clinical characteristics, well-annotated clinical response, and patient outcomes were used. From the discovery study, a reactive stroma signature was identified to be specifically associated with the platinum-resistant (Plat-R) primary tumors and was further up-regulated in Plat-R recurrent tumors. This signature was further validated in an independent data set and the clinical utility in predicting patient outcome for front-line platinum-based chemotherapy was demonstrated. These findings provide a diagnostic strategy for identifying primary chemotherapy-resistant ovarian cancer patients and provide a biomarker-based test for predicting response to primary chemotherapy.

### Materials and Experimental Methods

### Patients and Tumor Specimens

This study consisted of two sets of ovarian patient cohorts for discovery and validation purposes, respectively. The discovery set consisted of 85 high-grade serous or endometrioid ovarian cancers from 58 patients. The clinical characteristics of these patients are described in Table 6 and represent typical clinical profiles of patients with high-grade epithelial ovarian cancer. All 58 patients were initially treated with combination platinum and taxane. Of these, 32 patients had primary platinum-resistant tumors (disease recurrence or progression within 6 months post completion of front-line platinum-based chemotherapy) and 26 patients had platinum sensitive tumors (no recurrence or progression within 12 months of front-line chemotherapy). Tumor specimens were collected prior to front-line chemotherapy from all patients. Twenty-seven of the 32 platinum resistant patients also had patient matched tumor specimens collected at the time of recurrent disease. All discovery set tissue samples were obtained from commercial sources and had appropriate institutional approval.

The validation set consisted of 138 high-grade serous or endometrioid ovarian cancers from 138 patients from the chemotherapy treatment arm of a phase III trial, examining the effects of standard chemotherapy versus adding bevacizumab to standard chemotherapy in women with newly diagnosed ovarian cancer. The clinical characteristics of these patients are described in Table 9.

All tumor tissues were subjected to review by a pathologist to confirm diagnosis and tumor content. Macro-dissection was performed on formalin-fixed and paraffin embedded (FFPE) tumor tissue to enrich tumor percentage to greater than 70%. Total RNA was purified using High Pure FFPE RNA Micro Kits (Roche Diagnostics, Indianapolis, Ind., USA). FFPE tumor DNA was prepared using QIAamp DNA FFPE Tissue Kits (Qiagen, CA).

Gene Expression Profiling using an Ovarian Cancer Biomarker Nanostring Panel

A custom NanoString 800 GX CodeSet was designed to measure gene expression of 800 biomarkers and controls that are associated with ovarian disease biology, including subtype and prognosis classifiers, efflux ABC transporters, as well as chemo-tolerance, immune, and angiogenesis markers (see Table 5 for complete gene list). 200 ng RNA was analyzed using the NanoString nCounter Analysis System following the manufacturer's protocol (NanoString Technologies). Output raw counts were normalized by the median counts of all 800 assays for each sample.

TABLE 5
Complete gene list
AADAC
ABCA1
ABCA10
ABCA13
ABCA2
ABCA3
ABCA7
ABCA8
ABCB1
ABCB10
ABCB6
ABCB7
ABCB8
ABCB9
ABCC1
ABCC3
ABCC4
ABCC6
ABCC9
ABCD1
ABCD3
ABCG1
ABCG2
ACKR3
ACOT13
ACTA2
ACTB
ACTR3B
ACVRL1
ADAMDEC1
ADCK3
ADIPOR2
ADRM1
AGFG2
AGR2
AHNAK2
AIM2
AKAP12
AKT1
AKT2
AKT3
ALDH1A1
ALDH5A1
ALG13
ALPP
ALS2CL
ANGPT1
ANGPT2
ANGPTL1
ANGPTL2
ANLN
ANXA1
ANXA4
APEX1
APH1B
APLN
APOA1
APOBEC3G
AREG
ARF5
ASAP3
ATAD2
ATM
ATR
AURKA
AURKB
AXIN2
B4GALT5
BACE2
BAD
BAG1
BAMBI
BAX
BBC3
BCAT1
BCL2
BCL2L1
BCL2L11
BEX1
BGN
BIRC5
BLCAP
BLMH
BLVRA
BMP4
BNIP3
BRAF
BRCA1
BRCA2
BST2
BTG2
BTLA
C11orf30
C12orf5
C1orf116
C2CD2L
CA9
CACNA1C
CALD1
CASP1
CAV1
CCL2
CCL21
CCL22
CCL28
CCL3
CCL5
CCNA2
CCNB1
CCND1
CCND2
CCNE1
CCR5
CCR7
CD14
CD163
CD1C
CD247
CD27
CD274
CD276
CD28
CD36
CD38
CD3D
CD3E
CD4
CD40
CD40LG
CD44
CD47
CD48
CD68
CD69
CD70
CD79B
CD80
CD86
CD8A
CDC2
CDC20
CDC25B
CDC25C
CDC42
CDC6
CDCA7L
CDCA8
CDH1
CDH2
CDH3
CDH5
CDH6
CDK1
CDK4
CDKN1A
CDKN1C
CDKN2A
CDKN3
CEACAM5
CENPE
CENPF
CEP55
CH25H
CHEK1
CHEK2
CHIT1
CHMP4C
CIITA
CITED2
CKS1B
CLDN3
CLDN4
CLDN5
CLDN6
CLEC14A
CLEC5A
CLU
COL15A1
COL18A1
COL4A1
COL4A2
COL4A5
COL4A6
COL5A1
COL8A1
COL9A1
COPS3
CPE
CRB3
CRYAB
CSF1
CSF1R
CSF2
CSNK1A1
CST6
CTGF
CTLA4
CTNNB1
CTNNBL1
CTPS2
CUTA
CX3CL1
CXCL1
CXCL10
CXCL11
CXCL12
CXCL13
CXCL2
CXCL9
CXCR3
CXCR4
CXXC5
CYFIP2
CYR61
CYTH3
DAP
DDB2
DDIT4
DDR2
DLC1
DLGAP4
DLL4
DNAJB5
DTX4
DUSP4
DUSP6
E2F6
EBNA1BP2
ECH1
EDNRB
EFNB2
EFS
EGFL7
EGFR
EIF3K
EIF4A1
EIF4B
ELF4
ELTD1
EMCN
ENG
ENPP3
EOMES
EPCAM
EPHA4
EPHB4
ERBB2
ERBB3
ERBB4
ERCC1
ESM1
ESR1
ESR2
ETS1
EVI2A
EXO1
EXOC6B
EZH1
F2R
FAM111A
FAM174A
FAM198B
FAM214A
FAM8A1
FANCA
FANCD2
FANCF
FAP
FASN
FBLIM1
FBLN1
FBXL18
FBXO5
FBXW7
FCER1G
FCRL5
FGF1
FGF2
FGFR1
FGFR2
FGFR3
FGFR4
FJX1
FLT1
FN1
FOLR1
FOS
FOSL1
FOXA1
FOXA2
FOXC1
FOXC2
FOXM1
FOXO1
FOXO3
FOXP3
FSCN1
FUT2
FXYD2
FYN
FZD5
G6PD
GAD1
GADD45A
GALNT10
GAPDH
GAS6
GAS7
GBP1
GCNT1
GCNT1
GCNT1
GDF15
GFRA1
GGH
GIMAP5
GIPC1
GJB1
GLDC
GLS
GMPR
GOT1
GPC3
GPC4
GPM6B
GPR160
GPRC5A
GSTM1
GTF2F2
GUCY1B3
GUSB
GZMA
GZMB
GZMK
HAVCR2
HBEGF
HDAC1
HDAC4
HES1
HEY1
HGF
HHEX
HIF1A
HLA-A
HLA-DOB
HLA-E
HMGA2
HMMR
HNF1B
HOXA10
HOXA11
HOXA5
HOXA7
HOXA9
HOXC6
HOXD10
HSP90AA1
HSPA13
HSPA1L
HSPB7
ICAM1
ICAM2
ICOS
ID1
IDO1
IF116
IF130
IFNG
IGF1R
IGFBP2
IGFBP3
IGFBP7
IGSF3
IL10
IL12A
IL17A
IL1B
IL21R
IL2RA
IL6
IL7R
IL8
INADL
INSIG1
INSR
IRF2BP1
IRS1
IRS2
ITGAM
ITGB6
JAG1
JAG2
JUN
KCNE3
KDELC1
KDM2B
KDM5A
KDM5B
KDR
KIAA0040
KIAA0247
KIAA1033
KIF1A
KIF23
KIF2C
KIF4A
KIFC1
KIT
KITLG
KLK6
KLK7
KLRK1
KRAS
KRT14
KRT17
KRT18
KRT19
KRT5
LAG3
LAIR1
LAMA4
LAMB1
LAPTM5
LCK
LCN2
LDHA
LDHB
LGALS1
LGALS3
LGALS3
LGALS4
LGALS8
LGALS9
LGR5
LIPC
LOX
LRIG1
LRP4
LUC7L2
LY6E
MAD2L1
MAML1
MAML2
MAML3
MAMLD1
MAN1A1
MAP2
MAP2K1
MAP2K2
MAP2K4
MAP3K5
MAP4K1
MAPK1
MAPK14
MAPK3
MAPK8
MAPRE1
MAPRE2
MARCH6
MARCKS
MARCKSL1
MARK4
MCAM
MCL1
MCM2
MCM3
MDM2
MECOM
MED16
MEF2C
MELK
MERTK
MEST
MET
MFAP2
MGAT5
MGLL
MGMT
MIA
MICA
MICB
MIS18A
MITF
MKI67
MLH1
MLPH
MMP10
MMP11
MMP12
MMP14
MMP3
MMP7
MMRN2
MRPS12
MS4A1
MSLN
MST1R
MTAP
MTCH1
MUC1
MUC16
MVP
MXRA8
MYBL2
MYC
MYCN
MYCT1
MYO1B
MYO5C
NANOG
NAT1
NBL1
NCAM1
NCAPH2
NDC80
NEBL
NEO1
NETO2
NF1
NFKB1
NFKBIB
NID1
NID2
NMI
NNMT
NOTCH1
NOTCH2
NOTCH3
NOTCH4
NPEPPS
NREP
NRG1
NRP1
NSG1
NT5E
NUAK1
NUDT1
NUF2
NUP98
OPA3
ORC6
PAGR1
PAK1
PAK4
PAK6
PALB2
PALLD
PARD6B
PARP1
PCDH12
PCDH17
PCNA
PCOLCE
PDCD1
PDCD1LG2
PDCD4
PDGFRA
PDGFRB
PDP1
PDPN
PDZK1IP1
PECAM1
PEX6
PGF
PGR
PHGDH
PHKA1
PHLDA1
PHLDA3
PHLPP2
PI3
PIK3CA
PIK3CB
PIK3CD
PIK3CG
PIK3IP1
PKIA
PLAU
PLEKHM1
PLEKHO1
PLK1
PLVAP
PMAIP1
PMEPA1
PMVK
PODXL
POLD1
POSTN
POU5F1
PPIA
PPP1R13L
PRAME
PREP
PREX2
PRF1
PRKDC
PROM1
PRSS16
PRSS2
PSAT1
PSMC4
PSTPIP1
PTEN
PTGER2
PTGER4
PTGS2
PTPRB
PTPRCCD45_all
PTPRCCD45RO
PTPRCCD45RA
PTTG1
PTTG1IP
QPRT
RAB25
RAB40B
RABEP2
RAC1
RAC2
RAD21
RAD51
RAD51AP1
RAD51C
RAE1
RAF1
RARRES2
RARRES3
RASGRP3
RASIP1
RASSF1
RB1
RBP4
RBP7
RECK
RERG
RET
RFC1
RFC4
RGL2
RGS1
RGS5
RHOBTB3
RHOJ
RIN1
RND3
RNF103
RNF125
ROBO4
RORC
RPS16
RPS6KA1
RPS6KA2
RRM1
RRM2
RUNX1
RUNX3
RXRB
S100A10
S100A9
SALL2
SAMD4B
SAMSN1
SASH1
SCD
SDF2L1
SEMA6A
SERPINF1
SFRP1
SFRP4
SH3PXD2A
SIRT5
SKP1
SLA
SLC2A1
SLC31A2
SLC34A2
SLC37A1
SLC37A4
SLC39A6
SLC3A1
SLC4A4
SLC7A11
SLIT2
SLPI
SMARCD1
SNAI1
SNAI2
SNCA
SNRPA1
SOD2
SORL1
SOX11
SOX18
SOX2
SP2
SPARC
SPARCL1
SPATS2
SPDEF
SPRY2
SPRY4
SRC
SREBF2
SRGN
SRPX2
SSH3
ST6GAL1
STAT1
STAT3
STAT5A
STEAP1
STEAP3
STMN1
SUMO1
SUPT5H
TAP1
TBX21
TC2N
TCEAL1
TCF15
TCF7L1
TDO2
TFF1
TFPI2
TFRC
TGFB1
THBS1
TIAM1
TIGIT
TIMP1
TIMP3
TJP3
TLCD1
TMEFF1
TMEM30B
TMEM45B
TMEM55B
TMEM88
TMPRSS4
TNF
TNFRSF14
TNFRSF4
TNFRSF9
TNFSF4
TNFSF9
TOP1
TOP2A
TOX
TP53
TP53TG5
TP63
TP73
TPST1
TRIM27
TRIP13
TRO
TSC1
TSC2
TSPAN8
TTF1
TTPAL
TUBA4A
TUBB2A
TWIST1
TXNDC5
TYMP
TYMS
TYRO3
UBD
UBE2C
UBE2L6
UBE2T
UCHL1
UNC5B
URI1
UTP20
VCAM1
VEGFA
VEGFB
VEGFC
VIM
VPS33B
VPS52
VTCN1
WAS
WBP4
WDR45B
WDR77
WFDC2
WIPF1
WNT2
XIAP
XPO4
ZC3H13
ZEB1
ZEB2
ZFHX4
ZMAT3
ZNF12
ZNF76
ZNF780B

### Statistical Analysis

Progression-free survival was calculated from the date of randomization to the date of the first indication of disease progression or death, whichever occurred first; the data for patients who were alive without disease progression were censored as of the date of their last non-progressive disease (PD) tumor assessment. Overall survival was calculated from the date of randomization to the date of death from any cause; data for patients still alive were censored at the date the patient was last known to be alive. Survival analysis was carried out using log-rank test for the difference in the distribution of progression-free survival between the biomarker high and low groups. Median survival time was computed using the product-limit estimate by the Kaplan Meier method.

To compare gene expression differences between Plat-S and Plat-R primary tumors, two-sample t tests were employed. To compare gene expression differences between Plat-R matched primary and metastatic tumors, paired t tests were used. Two-sided p values were derived and adjusted for multiple comparisons by controlling for false discovery rate (FDR) using the Benjamini Hochberg method.

### RNA In Situ Hybridization (RNA ISH) Assays

Duplex POSTN/LOX and single-plex FAP RNAscope® in situ hybridization (ISH) assays were designed, implemented, and scored at Advanced Cell Diagnostics, Hayward, Calif. The single color probe for FAP (NM_004460.2, nt 237-1549) was pre-designed and commercially available. Dual color paired double-Z oligonucleotide probes were designed against LOX (GenBank accession number NM_001178102.1, nt 223-1725) and POSTN (NM_006475.2, nt 13-1199) RNAs, using custom software as described in Wang et al., J Mol Diagn 14:22-29 (2012). RNA ISH was performed using the RNAscope® 2-plex Chromogenic Reagent Kit and RNAscope® 2.0 HD Brown Reagent Kit on 4 μm formalin-fixed, paraffin-embedded (FFPE) tissue sections according to the manufacturer's instructions. RNA quality was evaluated for each sample with a dual colored probe specific to the housekeeping gene cyclophilin B (PPIB) and RNA polymerase subunit IIA (PolR2A). Negative control background staining was evaluated using a probe specific for the bacterial dapB gene. Only samples with an average of >4 dots per cell with the housekeeping gene probe staining, and an average of <1 dot per 10 cells with the negative control staining, were assayed with target probes. To verify technical and scoring accuracy, reference slides consisting of FFPE HeLa cell pellets were tested for PPIB and dapB together with tissue FFPE slides. Bright field images were acquired using a Zeiss Axio Imager M1 microscope using a 40× objective. The RNAscope signal was scored based on the number of dots per cell as follows 0=0 dots/cell, 1=1-3 dots/cell, 2=4-9 dots/cell, 3=10-15 dots/cell, and 4=>15 dots/cell with >10% of dots in clusters. To evaluate heterogeneity in marker expression, H-score analysis was performed. The H-score was calculated by adding up the percentage of cells in each scoring category, multiplied by the corresponding score, so the scores are on a scale of 0-400.

### Immunohistochemistry

Immunohistochemistry (IHC) was performed on 4 μm thick formalin-fixed, paraffin-embedded tissue sections mounted on glass slides. Primary antibodies against FAP (GNE, clone 10D2.1.1), alpha smooth muscle actin (SMA) (AbCam, Cambridge, Mass.), and POSTN (BioVendor, Asheville, N.C.) were used. FAP staining was performed on the DAKO autostainer, utilizing Trilogy (Cell Marque, Rocklin, Calif.) antigen retrieval. Detection employed horse anti-mouse biotinylated secondary antibody (VectorLabs, Burlingame, Calif.), followed by Streptavidin-HRP with TSA enhancement (PerkinElmer, Waltham, Mass.) and DAB visualization (Pierce, Rockford, Ill.). SMA and POSTN staining was carried out on the Ventana Discovery XT automated platform (Ventana Medical Systems; Tucson, Ariz.). Sections were treated with Cell Conditioner 1, standard time. Specifically bound primary antibody was detected by incubating sections in OmniMap anti-Rabbit-HRP (Ventana Medical Systems; Tucson, Ariz.) followed by ChromoMap DAB (Ventana Medical Systems; Tucson, Ariz.). The sections were counterstained with hematoxylin, dehydrated, and coverslipped.

### H&E Assessment of Desmoplasia

Representative H&E stained sections of the discovery tumor samples (85 total including primary Plat-S, patient-matched Plat-R primary, and recurrent tumors) were examined for evidence of stromal activation associated with tumor insult and a desmoplasia score was assigned. Some cases were deemed too difficult to score on the representative section available due to tissue damage, necrosis, edema, or limited stroma present. Desmoplasia were identified as fibrotic regions typified by an increased density and disorganization of myofibroblasts distinct from resident non-activated fibroblasts. The desmoplasia scoring system used is similar to that reported by Tothill et al., Clin Cancer Res. 14:5198-5298, 2008. Desmoplasia scores were defined as follows: 0=no desmoplasia, 1=few scattered desmoplastic foci abutting cancer cells, 2=several desmoplastic foci abutting cancer cells or moderate confluent (wider) desmoplasia, but not present throughout the section, 3=desmoplastic reaction throughout section.

### TP53 Mutation Status

Deep sequencing was performed on all exons and exon-intron junctions of the entire TP53 gene using a previously developed MMP-Seq targeted cancer panel. Quality of the FFPE DNA samples was quantified as number of functional copies using a TRAK2 qPCR “ruler assay.” 5000 functional copies of DNA from each sample were used as the input for target enrichment and library construction using Fluidigm Access Arrays followed by deep sequencing on an Illumina MiSeq sequencer. The average coverage of the TP53 gene was ˜1000× per amplicon. Sequence alignment, primary variant calling, and filtering was performed as described in Bourgon et al., Clin Cancer Res 20:2080-2091 (2014).

### Copy Number Variation Analysis by Real-Time PCR

Genomic formalin-fixed paraffin embedded (FFPE) DNA (200ng) was subjected to 17 cycles of pre-amplification using a pool of 35 pairs of gene specific primers at 50 nM each and Taqman Preamplification Master Mix (Life Technologies) according to the manufacture's protocol. The preamplified samples were diluted and qPCR was performed using the Fluidigm 96.96 Dynamic Arrays on the BioMark™ system. In brief, sample mix contained DNA, Taqman gene Expression Master Mix (Life Technologies), DNA binding sample loading reagent (Fluidigm), and EvaGreen dye (Biotium). The assay mix contained gene specific primer pairs and sample loading reagent (Fluidigm). The Ct determination and melting curve analyses were carried out using Fluidigm gene analysis software. Relative gene copy numbers were calculated using the global Delta Delta Ct method. First, the median Ct of all genes in each sample was used as reference to normalize sample DNA input and calculate the delta Ct. The median delta Ct of all samples for individual genes was then used as a 2 copy calibrator sample. Results are the average of three primer pairs for each gene.

### Cell-Based Assays

Ovarian cell line ES-2 was obtained from the ATCC and cultured in RPMI1640 medium with 10% FBS and 2 mM glutamine. 96-well plates were first coated with recombinant full-length FN1 (Cat #F2006, Sigma-Aldrich, St. Louis, Mo.), POSTN (Cat #3548-F2, R&D Systems, Minneapolis, Minn.), or left uncoated at 37° C. for 2 hours or 4° C. for 16 hours. Cells were then plated in coated plates at 3,000 cells/well. 10 μM carboplatin or 10 nM paclitaxel was added to each well on the next day. Cell-Titre Glo® reagents were added at 72 hours after compound treatment to measure cell viability. The viability in coated wells was then compared with the viability in uncoated wells to calculate % growth benefit.

### Example 1

### Identification of a “Reactive Stroma” Gene Signature that is Up-Regulated in Primary Chemotherapy-Resistant Ovarian Tumors

To identify molecular characteristics associated with primary chemotherapy-resistance in EOC, a set of high-grade serous or endometrioid ovarian tumors with clinically well-defined response to primary chemotherapy were selected (Table 6). This discovery set consisted of tumor specimens from 32 patients with primary chemotherapy-resistance and 26 patients who were sensitive to primary chemotherapy. All patients were treated with a combination of platinum and taxane as front-line chemotherapy. Primary chemotherapy-resistant patients were selected based on having had disease recurrence or progression within 6 months post completion of the front-line platinum-based chemotherapy, while chemotherapy-sensitive patients were selected based on having had no recurrence or progression within 12 months from primary chemotherapy. 27 out of 32 chemotherapy-resistant patients had patient-matched primary tumor specimens collected prior to chemotherapy and recurrent tumor specimens collected post therapy at disease progression (referred to as Plat-R primary and Plat-R recurrent, respectively). For the 26 chemotherapy-sensitive patients, only primary tumor specimens prior to therapy were available for analysis (referred to as Plat-S primary).

TABLE 6
Patient clinicopathological characteristics in the discovery study

| | Platinum- | Platinum- |
|---|---|---|
| | Resistant (N = 32) | Sensitive (N = 26) |
| Age: Median (range) | 56 (28-76) | 47.5 (28-64) |
| Stage: | | |
| I | 1 (3.1%) | 6 (23.1%) |
| II | — | — |
| III | 31 (96.9%) | 20 (76.9%) |
| IV | — | — |
| Histology: | | |
| Serous | 30 (93.8%) | 25 (96.2%) |
| Endometrioid | 2 (6.2%) | 1 (3.8%) |
| PFI (Platinum-Free | | |
| Interval): | | |
| From end of primary TX | 4.4 | Not Reached |
| Median (months) | (3.9, 5.0) | (NA, NA) |
| 95% confidence interval | 32 | 0 |
| events | | |
| OS | | |
| From surgery | 21.9 | Not Reached |
| Median (months) | (20.0, 31.5) | (NA, NA) |
| 95% confidence interval | 25 | 0 |
| events | | |

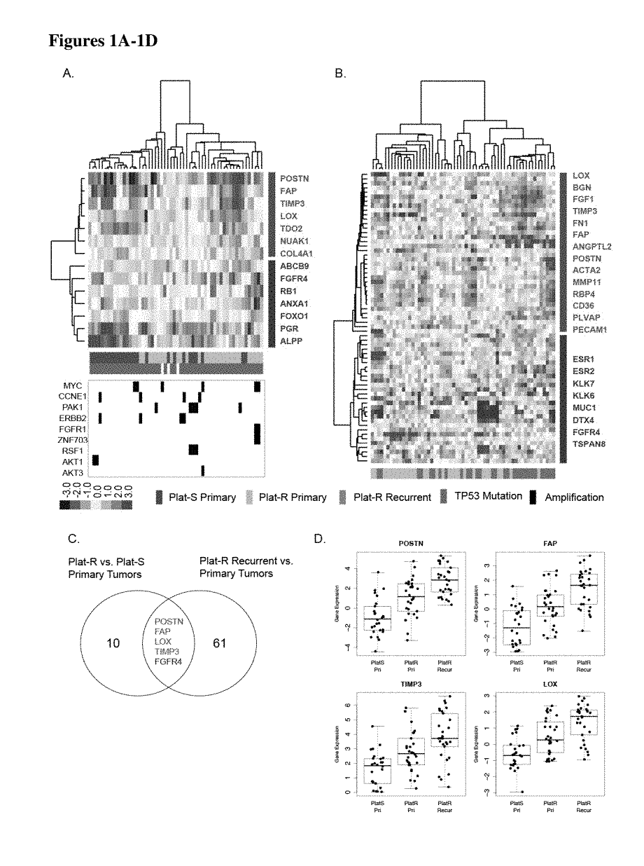

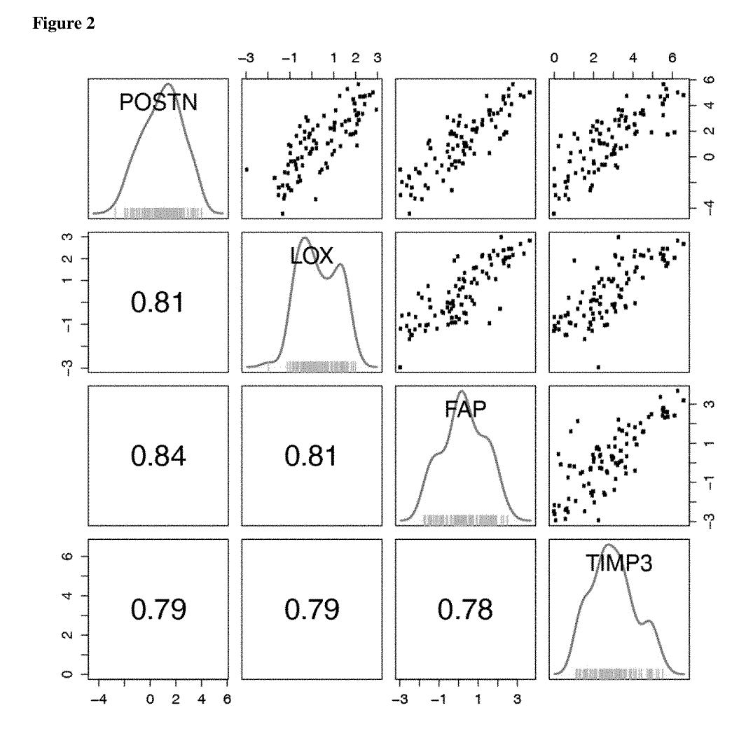

A gene expression signature that correlates with responses to platinum-based chemotherapy was sought. Gene expression profiling was performed on the Plat-R primary, Plat-R recurrent, and Plat-S primary samples using an 800-gene ovarian cancer biomarker panel (Table 5) developed on the Nanostring platform. Two-sample t tests comparing 32 Plat-R and 26 Plat-S primary tumors prior to chemotherapy identified 14 genes that are significantly differentially expressed between the two groups (FDR ≦10% and fold change ≧1.5, Table 7). Up-regulated genes in the Plat-R tumors represented a distinct “reactive stroma” signature (FIG. 1A), highly enriched in ECM production and remodeling genes (i.e., POSTN, FAP, LOX, TIMP3, COL4A1), genes involved in cell migration and invasion (i.e., NUAK1), as well as genes involved in immune modulation (i.e., TDO2). On the other hand, key genes associated with chemotherapy-sensitive tumors include progesterone receptor (PGR), placental alkaline phosphatase (ALPP), and fibroblast growth factor 4 (FGFR4) genes. For the 27 Plat-R patients who had patient-matched primary tumor specimens collected prior to therapy and recurrent tumor specimens collected post therapy at disease progression, further analysis was performed to search for gene signatures characterizing recurrent tumors. Paired t-test identified 65 genes that were significantly differentially expressed between the primary and recurrent resistant tumors (FDR <10% and fold change >1.5, Table 8). Again, hallmark genes representing tumor stromal components were highly enriched among the 36 significantly up-regulated genes in the recurrent tumors (FIG. 1B), including an activated fibroblast marker (ACTA2), ECM production and remodeling enzymes (i.e., POSTN, FAP, FN1, TIMP3, LOX, MMP11), growth factors (i.e., FGF1), immune related genes (i.e., CD36, GZMK, CD247), as well as vascular endothelial markers (i.e., PLVAP and PECAM (antigen CD31)) and growth factors (i.e., ANGPL2). As compared to the primary tumors prior to therapy, the 29 significantly down-regulated genes in recurrent Plat-R tumors were estrogen receptors (ESR1 and ESR2) and other differentiated epithelial cell markers (MUC1, KLK6, KLK7) (FIG. 1B). Comparison of the two signatures characterizing primary and recurrent Plat-R tumors identified 4 common reactive stroma signature genes, POSTN, FAP, TIMP3, and LOX having expression levels that were (1) highly correlated with each other (FIG. 2); (2) significantly up-regulated in Plat-R primary tumors as compared to Plat-S primary tumors, and (3) further induced post chemotherapy treatment in Plat-R recurrent tumors (FIGS. 1C and 1D). Together, these results indicated that up-regulation of reactive stroma genes may play important roles in modulating chemotherapy-resistance in EOC.

Mutations in tumor suppressor gene TP53 and amplification of cyclin E1 (CCNE1) have been previously associated with primary chemotherapy-resistance in ovarian cancer. Deep sequencing was performed on all exons of the entire TP53 gene using the MMP-Seq targeted cancer panel. TP53 mutations were found in 32 out of 32 (100%) Plat-R primary tumors and 23 out of 26 (88%) Plat-S primary tumors (FIG. 1A). The observed overall high frequency of the TP53 mutation was consistent with TCGA findings in high-grade serous ovarian tumors. These results also indicated that TP53 mutation status was not likely to be the main driver in determining responses to chemotherapy treatment. A qPCR-based copy number analysis was also performed on 35 genes that have been reported to be frequently altered in many types of cancer. Nine recurrently amplified genes were identified in this study (FIG. 1A, copy number ≧4). Among these, RSF1, AKT1, and AKT3 amplification were only identified in Plat-S tumors, while FGFR1 and ZNF703 amplification were only identified in Plat-R tumors. However, no significant correlation was observed between response to chemotherapy and amplification of any one (including CCNE1) or combination of these genes.

TABLE 7
14 differentially expressed genes between Plat-R primary vs. Plat-S
primary tumors (discovery dataset)

| | Mean Fold Change in | | | |
|---|---|---|---|---|
| | Plat-R primary | Up or | | |
| | vs. Plat-S primary | Down in | | |
| Gene | tumors | PlatR | P Value | FDR |
| RB1 (Gene ID | −1.76403 | Down | 0.00011 | 0.02890 |
| No.: 5925) | | | | |
| TDO2 (Gene | 2.17582 | Up | 0.00021 | 0.02890 |
| ID No.: 6999) | | | | |
| POSTN (Gene | 4.00402 | Up | 0.00022 | 0.02890 |
| ID No.: 10631) | | | | |
| FAP (Gene ID | 2.62089 | Up | 0.00025 | 0.02890 |
| No.: 2191) | | | | |
| COL4A1(Gene | 1.66375 | Up | 0.00029 | 0.02890 |
| ID No.: 1282) | | | | |
| LOX(Gene ID | 2.08455 | Up | 0.00033 | 0.02902 |
| No.: 4015) | | | | |
| FGFR4 (Gene | −2.06441 | Down | 0.00052 | 0.03910 |
| ID No.: 2264) | | | | |
| PGR(Gene ID | −3.25575 | Down | 0.00056 | 0.03910 |
| No.: 5241) | | | | |
| TIMP3 (Gene | 2.31509 | Up | 0.00100 | 0.05864 |
| ID No.: 7078) | | | | |
| NUAK1 (Gene | 1.59941 | Up | 0.00101 | 0.05864 |
| ID No.: 9891) | | | | |
| ABCB9(Gene | −1.61825 | Down | 0.00115 | 0.06145 |
| ID No.: 23457) | | | | |
| FOXO1(Gene | −1.56584 | Down | 0.00147 | 0.07300 |
| ID No.: 2308) | | | | |
| ALPP (Gene | −3.29452 | Down | 0.00174 | 0.07989 |
| ID No.: 250) | | | | |
| ANXA1(Gene | −1.76479 | Down | 0.00195 | 0.07989 |
| ID No.: 301) | | | | |

TABLE 8
65 differentially expressed genes between Plat-R recurrent vs. Plat-R
primary tumors (discovery dataset)

| | | Up | | |
|---|---|---|---|---|
| | Mean Fold | or Down | | |
| | Change in Plat- | in Plat-R | | |
| | R recurrent vs. Plat-R | recurrent | | |
| Gene | primary tumors | tumors | P Value | FDR |
| DTX4 (Gene ID | −1.64787 | Down | 0.00002 | 0.01054 |
| No.: 23220) | | | | |
| CD36(Gene ID | 3.56536 | Up | 0.00003 | 0.01054 |
| No.: 948) | | | | |
| PLVAP (Gene | 1.84394 | Up | 0.00013 | 0.02312 |
| ID No.: 83483) | | | | |
| ESR2 (Gene ID | −2.00550 | Down | 0.00023 | 0.02412 |
| No.: 2100) | | | | |
| POSTN (Gene | 3.28556 | Up | 0.00029 | 0.02412 |
| ID No.: 10631) | | | | |
| KRT18 (Gene | −1.52693 | Down | 0.00032 | 0.02412 |
| ID No.: 3875) | | | | |
| ABCC9 (Gene | 1.73344 | Up | 0.00034 | 0.02412 |
| ID No.: 10060) | | | | |
| PCOLCE (Gene | 1.66209 | Up | 0.00039 | 0.02412 |
| ID No.: 5118) | | | | |
| FUT2 (Gene ID | −1.49515 | Down | 0.00041 | 0.02412 |
| No.: 2524) | | | | |
| CD1C (Gene ID | 1.73641 | Up | 0.00046 | 0.02412 |
| No.: 911) | | | | |
| MS4A1 (Gene | 2.63163 | Up | 0.00050 | 0.02412 |
| ID No.: 931) | | | | |
| CD44 (Gene ID | 1.59338 | Up | 0.00052 | 0.02412 |
| No.: 960) | | | | |
| ANGPTL2 | 1.55443 | Up | 0.00066 | 0.02412 |
| (Gene ID No.: | | | | |
| 23452) | | | | |
| PECAM1 (Gene | 1.56963 | Up | 0.00075 | 0.02412 |
| ID No.: 5175) | | | | |
| HOXD10 (Gene | −1.94235 | Down | 0.00081 | 0.02412 |
| ID No.: 3236) | | | | |
| FAP (Gene ID | 2.35907 | Up | 0.00088 | 0.02412 |
| No.: 2191) | | | | |
| LOX (Gene ID | 1.89374 | Up | 0.00103 | 0.02412 |
| No.: 4015) | | | | |
| TIMP3(Gene ID | 2.16769 | Up | 0.00107 | 0.02412 |
| No.: 7078) | | | | |
| EXO1 (Gene ID | −1.62390 | Down | 0.00108 | 0.02412 |
| No.: 9156) | | | | |
| INADL (Gene | −1.53801 | Down | 0.00109 | 0.02412 |
| ID No.: 10207) | | | | |
| PMEPA1 (Gene | 1.50167 | Up | 0.00113 | 0.02412 |
| ID No.: 56937) | | | | |
| IGFBP2 (Gene | −1.61594 | Down | 0.00113 | 0.02412 |
| ID No.: 3485) | | | | |
| IL7R (Gene ID | 2.04198 | Up | 0.00117 | 0.02412 |
| No.: 3575) | | | | |
| FBLN1 (Gene ID | 1.88186 | Up | 0.00130 | 0.02591 |
| No.: 2192) | | | | |
| FGF1 (Gene ID | 1.77319 | Up | 0.00135 | 0.02600 |
| No.: 2246) | | | | |
| RBP4 (Gene ID | 2.89945 | Up | 0.00141 | 0.02600 |
| No.: 5950) | | | | |
| TWIST1 (Gene | 1.52597 | Up | 0.00159 | 0.02600 |
| ID No.: 7291) | | | | |
| KLK7 (Gene ID | −1.73811 | Down | 0.00171 | 0.02600 |
| No.: 5650) | | | | |
| MYCN (Gene ID | −1.59335 | Down | 0.00183 | 0.02600 |
| No.: 4613) | | | | |
| FGFR4 (Gene ID | −1.65482 | Down | 0.00184 | 0.02600 |
| No.: 2264) | | | | |
| ID1 (Gene ID | 1.53481 | Up | 0.00187 | 0.02600 |
| No.: 3397) | | | | |
| ERBB3 (Gene | −1.50105 | Down | 0.00224 | 0.02737 |
| ID No.: 2065) | | | | |
| RAC2 (Gene ID | 1.67853 | Up | 0.00257 | 0.03030 |
| No.: 5880) | | | | |
| GFRA1 (Gene | 1.76644 | Up | 0.00286 | 0.03215 |
| ID No.: 2674) | | | | |
| TMEM45B | −1.65581 | Down | 0.00296 | 0.03218 |
| (Gene ID No.: | | | | |
| 120224) | | | | |
| MAN1A1(Gene | 1.58276 | Up | 0.00369 | 0.03537 |
| ID No.: 4121) | | | | |
| PROM1(Gene ID | −1.73404 | Down | 0.00377 | 0.03547 |
| No.: 8842) | | | | |
| NCAM1 (Gene | −1.79762 | Down | 0.00433 | 0.03821 |
| ID No.: 4684) | | | | |
| EVI2A (Gene ID | 1.66289 | Up | 0.00476 | 0.04087 |
| No.: 2123) | | | | |
| MKI67 (Gene ID | −1.50709 | Down | 0.00488 | 0.04091 |
| No.: 4288) | | | | |
| KLK6 (Gene ID | −1.55987 | Down | 0.00516 | 0.04194 |
| No.: 5653) | | | | |
| CCR7(Gene ID | 1.71160 | Up | 0.00555 | 0.04194 |
| No.: 1236) | | | | |
| CDH3 (Gene ID | −1.49953 | Down | 0.00560 | 0.04194 |
| No.: 1001) | | | | |
| LY6E (Gene ID | −1.50727 | Down | 0.00641 | 0.04601 |
| No.: 4061) | | | | |
| TJP3 (Gene ID | −1.59144 | Down | 0.00656 | 0.04611 |
| No.: 27134) | | | | |
| SLC7A11 (Gene | −1.69153 | Down | 0.00788 | 0.05192 |
| ID No.: 23657) | | | | |
| GZMK (Gene ID | 1.71790 | Up | 0.00958 | 0.05777 |
| No.: 3003) | | | | |
| TSPAN8 (Gene | −2.53992 | Down | 0.00963 | 0.05777 |
| ID No.: 7103) | | | | |
| BNIP3 (Gene ID | −1.54514 | Down | 0.01022 | 0.05854 |
| No.: 664) | | | | |
| PRAME (Gene | −1.63296 | Down | 0.01074 | 0.05980 |
| ID No.: 23532) | | | | |
| ESM1 (Gene ID | −1.64805 | Down | 0.01126 | 0.06107 |
| No.: 11082) | | | | |
| VTCN1 (Gene | −1.63373 | Down | 0.01158 | 0.06107 |
| ID No.: 79679) | | | | |
| PTPRC/CD45R | 1.74707 | Up | 0.01232 | 0.06131 |
| A (Gene ID No.: | | | | |
| 5788) | | | | |
| FCRL5 (Gene ID | 1.51619 | Up | 0.01289 | 0.06257 |
| No.: 83416) | | | | |
| ESR1 (Gene ID | −1.51432 | Down | 0.01297 | 0.06257 |
| No.: 2099) | | | | |
| MUC1 (Gene ID | −1.58715 | Down | 0.01547 | 0.06687 |
| No.: 4582) | | | | |
| NNMT (Gene ID | 1.57937 | Up | 0.01888 | 0.07640 |
| No.: 4837) | | | | |
| CCL28 (Gene ID | −1.52116 | Down | 0.01979 | 0.07872 |
| No.: 56477) | | | | |
| FN1 (Gene ID | 1.76729 | Up | 0.02084 | 0.08193 |
| No.: 633) | | | | |
| MMP11 (Gene | 1.82452 | Up | 0.02299 | 0.08743 |
| ID No.: 4320) | | | | |
| CD27 (Gene ID | 1.60143 | Up | 0.02341 | 0.08765 |
| No.: 939) | | | | |
| SLA (Gene ID | 1.50128 | Up | 0.02355 | 0.08765 |
| No.: 6503) | | | | |
| BGN (Gene ID | 1.50914 | Up | 0.02405 | 0.08765 |
| No.: 633) | | | | |
| ACTA2 ACTA2 | 1.54853 | Up | 0.02544 | 0.09035 |
| (Gene ID No.: | | | | |
| 59) | | | | |
| CD247 (Gene ID | 1.56026 | Up | 0.02941 | 0.09842 |
| No.: 919) | | | | |

### Example 2

### The Reactive Stroma Signature Genes are Derived and Modulated Specifically in Tumor Associated Fibroblasts

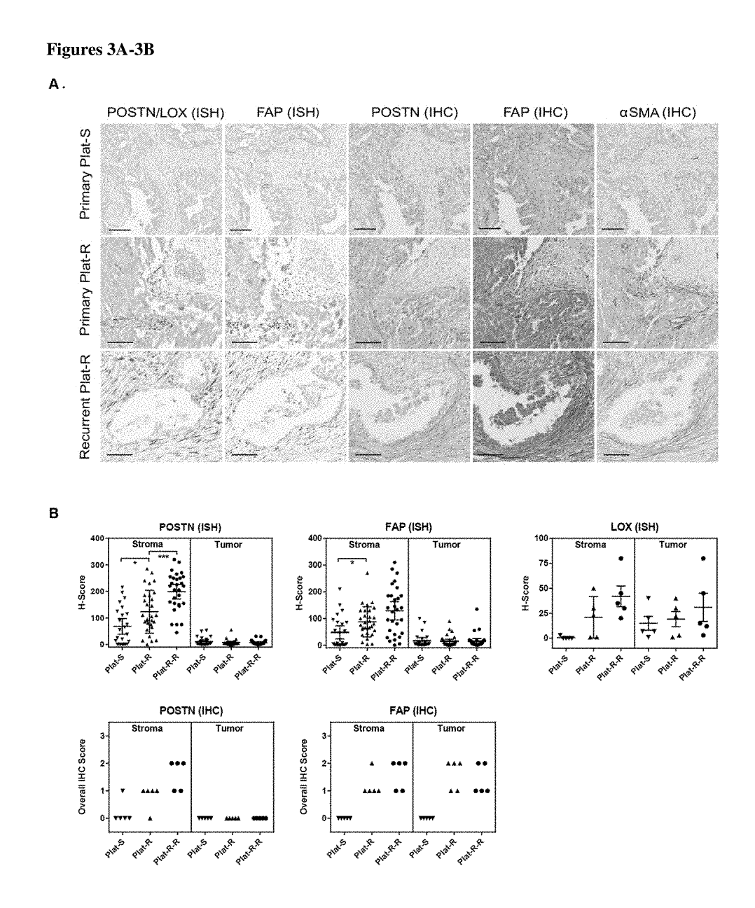

To determine which specific cell types expressed the reactive stroma signature genes, POSTN and FAP RNA ISH analysis was performed on whole slides of tumor specimens from the entire set of 85 tumor specimens. In addition, POSTN and FAP IHC, as well as LOX RNA ISH analysis were also performed on 15 representative tumor specimens. Representative images showing ISH and IHC of these markers are shown in FIG. 3A. In Plat-S primary tumors, none or significantly lower levels of the reactive stroma signature genes were detected in stromal or tumor cells by ISH or IHC. In contrast, in Plat-R primary and recurrent tumors, it was found that POSTN was exclusively expressed in the tumor-associated fibroblasts, while LOX and FAP were predominantly expressed in tumor-associated fibroblasts and at lower levels in tumor cells. The POSTN/LOX/FAP expressing tumor-associated fibroblasts also showed strong alpha-smooth muscle actin (αSMA) staining, which is an established marker for activated myofibroblasts. Consistent with the results from the Nanostring gene expression profiling (FIG. 1D), ISH and IHC analysis confirmed that expression of reactive stroma genes was significantly higher in Plat-R primary tumors compared to Plat-S primary tumors, and was further up-regulated in Plat-R recurrent tumors (FIG. 3B). The observed modulation of reactive stroma gene expression was mostly restricted to the stromal compartment immediately juxtaposed to the tumor cells in primary and recurrent Plat-R tumors (FIG. 3B), showing that the tumor-associated stromal compartments may be a specific site of action in mediating chemotherapy-resistance in ovarian cancer. Thus, using in situ analysis including both IHC and RNA ISH, the reactive stroma signature genes were identified as being exclusively or predominantly expressed by the activated fibroblast cells immediately juxtaposed to the tumor cells.

### Example 3

### Stromal Expression of POSTN is Associated with the Desmoplasia Phenotype

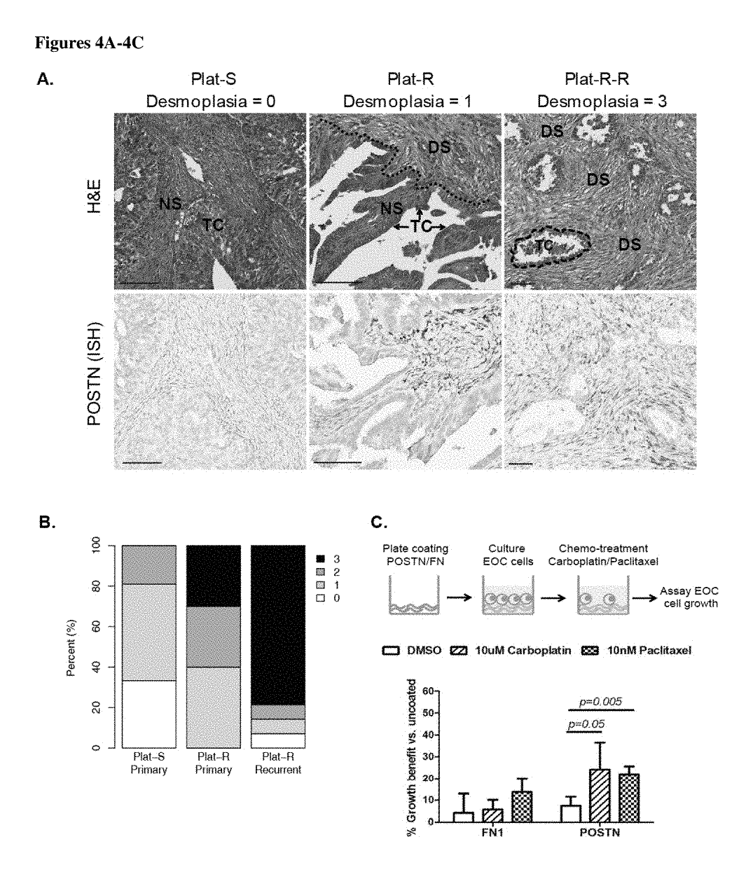

Desmoplasia is a common pathological phenotype found in many types of cancer. Histologic manifestations of desmoplasia include significant overproduction of extracellular matrix proteins, and extensive proliferation and disorganization of myofibroblast-like cells. Changes in stromal cell proliferation and the deposition of extracellular matrix components result in dramatic changes in overall tissue heterogeneity and elasticity, as well as accompanying interstitial fluid pressure. These changes have been suggested to contribute to chemotherapy-resistance in cancer. To evaluate potential links between the reactive stroma molecular signature and desmoplasia physiological features, the degree of desmoplasia was scored on H&E stained whole tissue sections for the entire set tumor specimens in this study. Of the 85 specimens that were scored, 26 of them were deemed too difficult to score due to tissue damage, necrosis, edema, or limited stroma present. The remaining specimens comprised 21 Plat-S primary, 18 Plat-R primary and 21 Plat-R recurrent tumors. As shown in FIG. 4A and 4B, while no or only a few scattered desmoplastic foci were observed in the majority of the Plat-S primary tumors, moderate to extensive desmoplasia were highly enriched in Plat-R primary and recurrent tumors. Furthermore, the degree of desmoplasia was highly correlated with stromal expression levels of POSTN, one of the key components of the reactive stroma signature characterizing primary chemotherapy-resistance. To further establish a direct role of these reactive stroma signature genes in mediating chemotherapy-resistance, it was demonstrated that chemotherapy-sensitive ovarian cells grown in the presence of recombinant POSTN became resistant to carboplatin and paclitaxel treatment in vitro.

### Example 4

### POSTN Promotes Chemotherapy-Resistance of EOC Cells In Vitro

Whether the reactive stroma signature genes play a specific role in promoting chemotherapy-resistance in ovarian tumor cells was next investigated. For this, recombinant human POSTN protein was used to coat tissue culture dishes to directly test its effects on resistance to chemo-reagents in ES-2 cells, a chemotherapy-sensitive ovarian cancer cell line with no endogenous POSTN expression (FIG. 4C). Because fibronectin (FN), a glycoprotein and key component of ECM, has been shown to modulate docetaxel resistance in ovarian cancer cells, FN protein coating was used as a control in this experiment. As shown in FIG. 4C, ES-2 cells grown on POSTN-coated plates were found to be significantly more resistant to carboplatin or paclitaxel treatment than cells grown on untreated culture dishes. Although POSTN coating alone also showed a small increase in cell growth in the absence of chemotherapy treatment, its effect on providing survival benefit upon chemotherapy treatment was predominant and significant. In contrast, FN coating provided much less of an effect on promoting drug resistance to carboplatin or paclitaxel treatment in ES-2 cells as compared to POSTN. This study demonstrated that POSTN can promote chemotherapy-resistance in EOC cells in vitro. Together, these results provided further supporting evidence that POSTN and other reactive stromal components may play a direct role in promoting chemotherapy-resistance in vivo.

### Example 5

### Independent Validation of the Reactive Stroma Signature in Association with Primary Chemotherapy-Resistance

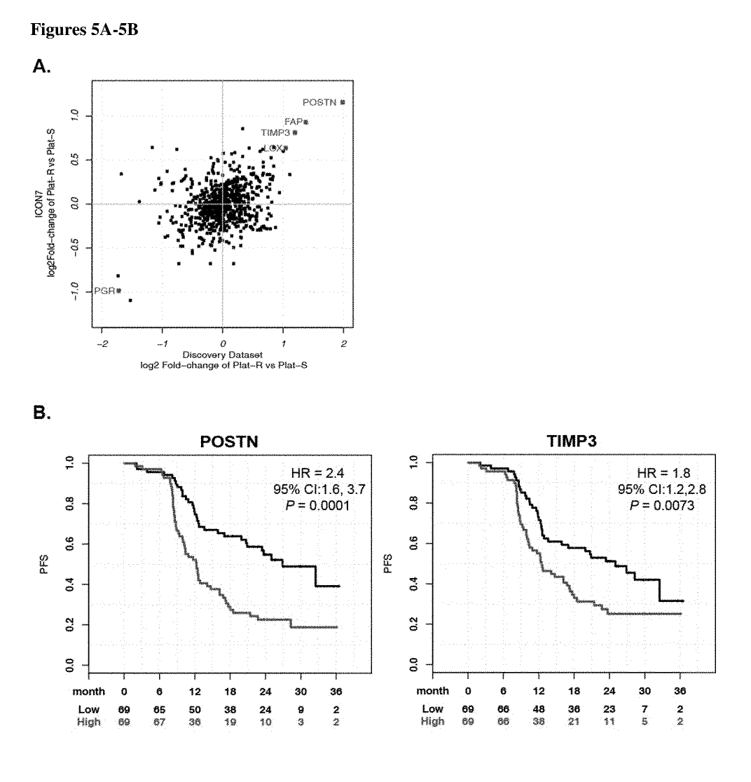

To further validate the direct link between the reactive stroma signature and primary chemotherapy-resistance in an independent dataset, a subset of ovarian tumor tissue samples were used from the chemotherapy treatment arm of a phase III trial evaluating the benefit of adding bevacizumab to standard chemotherapy as a front-line treatment of ovarian cancer (ICON7). Among the 510 patients enrolled in the chemo-control arm, 138 patients with high-grade serous or endometrioid ovarian tumors had tissue available for gene expression profiling on a Nanostring ovarian cancer biomarker panel (Table 9). No significant biases in terms of distribution of Plat-R and Plat-S patients, or clinicopathological characteristics were found in the biomarker subpopulation, suggesting it is representative of the intention-to-treat (ITT) population (Table 10). Patients from the chemo-control arm of the phase III trial were categorized into Plat-S and Plat-R groups using the same clinical definition used in the discovery study (Example 1, above). Two sample t-test analysis on 49 Plat-R and 86 Plat-S primary tumors prior to chemotherapy identified 10 genes that are significantly differentially expressed between the two groups (p≦0.01 and fold change ≧1.5, Table 11). Comparison of the differentially expressed gene lists from this dataset and the discovery dataset showed all four reactive stroma signature genes (POSTN, FAP, TIMP3, and LOX) constituting the top four significantly up-regulated genes in the primary chemotherapy-resistance tumors (FIG. 5A). These results independently confirmed that the reactive stroma signature is a robust and reproducible chemotherapy-resistance signature in EOC. Expression of PGR was consistently down-regulated by at least 2-fold in the chemotherapy-resistant group in both the discovery and the validation datasets (p<0.001 and fold change =3.3 in the discovery dataset; and p=0.0058 and fold change=2 in the validation dataset), suggesting that progesterone signaling may play an important role in mediating sensitivity to chemotherapies in ovarian cancer.

TABLE 9
Patient clinicopathological characteristics in the validation set from the
standard chemotherapy arm of a phase III clinical study

| | Platinum- | Platinum- |
|---|---|---|
| | Resistant (N = 37) | Sensitive (N = 67) |
| Age: Median (range) | 58 (43-79) | 58 (37-75) |
| Stage: | | |
| I | — | 4 (6%) |
| II | 2 (5.4%) | 11 (16.4%) |
| III | 27 (73%) | 52 (77.6%) |
| IV | 8 (21.6%) | — |
| Histology: | | |
| Serous | 33 (89.2%) | 59 (88.1%) |
| Endometrioid | 4 (10.8%) | 8 (11.9%) |
| PFI (Platinum-Free | | |
| Interval): | | |
| From end of primary TX | 4.6 | Not Reached |
| Median (months) | (4.5, 4.8) | (28.7, NA) |
| 95% confidence interval | 37 | 19 |
| events | | |
| OS | | |
| From surgery | 24.1 | Not Reached |
| Median (months) | (21.1, NA) | (NA, NA) |
| 95% confidence interval | 19 | 0 |
| events | | |

TABLE 10
Demographics summary of ICON7 chemo-treatment arm (biomarker
population vs. ITT)

| | All (ITT) | Biomarker |
|---|---|---|
| Age | | |
| N | 528 | 138 |
| Mean | 57.71 | 58.28 |
| SD | 10.28 | 9.4 |
| Median | 58 | 58 |
| Min-Max | 18 . . . 81 | 37 . . . 79 |
| ECOG PS | | |
| Total | 528 | 138 |
| 0 | 266 (50.38%) | 68 (49.28%) |
| 1 | 229 (43.37%) | 60 (43.48%) |
| 2 | 33 (6.25%) | 10 (7.25%) |
| Origin of Cancer | | |
| Total | 528 | 138 |
| FALLOPIAN TUBE | 21 (3.98%) | 3 (2.17%) |
| MULTIPLE LOCATIONS | 10 (1.89%) | 4 (2.9%) |
| OVARY (EPITHELIAL) | 456 (86.36%) | 124 (89.86%) |
| PRIMARY PERITONEAL | 41 (7.77%) | 7 (5.07%) |
| Histology | | |
| Total | 528 | 138 |
| CLEAR CELL | 0 (0%) | 0 (0%) |
| ENDOMETRIOID | 51 (9.66%) | 14 (10.14%) |
| MIXED | 0 (0%) | 0 (0%) |
| MUCINOUS | 0 (0%) | 0 (0%) |
| OTHER | 0 (0%) | 0 (0%) |
| SEROUS | 477 (90.34%) | 124 (89.86%) |
| Grade | | |
| Total | 528 | 138 |
| GRADE 1 | 0 (0%) | 0 (0%) |
| GRADE 2 | 119 (22.54%) | 28 (20.29%) |
| GRADE 3 | 409 (77.46%) | 110 (79.71%) |
| UNKNOWN | 0 (0%) | 0 (0%) |
| FIGO Stage | | |
| Total | 528 | 138 |
| IA | 6 (1.14%) | 0 (0%) |
| IB | 3 (0.57%) | 0 (0%) |
| IC | 14 (2.65%) | 5 (3.62%) |
| IIA | 8 (1.52%) | 1 (0.72%) |
| IIB | 18 (3.41%) | 4 (2.9%) |
| IIC | 23 (4.36%) | 9 (6.52%) |
| III | 13 (2.46%) | 4 (2.9%) |
| IIIA | 16 (3.03%) | 7 (5.07%) |
| IIIB | 30 (5.68%) | 8 (5.8%) |
| IIIC | 315 (59.66%) | 87 (63.04%) |
| IV | 82 (15.53%) | 13 (9.42%) |
| Debulking Surgery Residuum | | |
| Total | 528 | 138 |
| No Surgery | 9 (1.7%) | 1 (0.72%) |
| OPTIMAL | 363 (68.75%) | 85 (61.59%) |
| SUB-OPTIMAL | 156 (29.55%) | 52 (37.68%) |
| FIGO Stage and Residuum | | |
| Total | 528 | 138 |
| I-III with residual disease <=1 cm | 325 (61.55%) | 83 (60.14%) |
| I-III with residual disease >1 cm | 117 (22.16%) | 42 (30.43%) |
| IV and inoperable III | 86 (16.29%) | 13 (9.42%) |
| ITT Chemo | | |
| Total | 528 | 138 |
| <=4 weeks | 235 (44.51%) | 59 (42.75%) |
| >4 weeks | 293 (55.49%) | 79 (57.25%) |
| CA-125 | | |
| Total | 528 | 138 |
| <2x ULN | 199 (37.69%) | 66 (47.83%) |
| >=2x ULN | 322 (60.98%) | 71 (51.45%) |
| Missing | 7 (1.33%) | 1 (0.72%) |

TABLE 11
10 differentially expressed genes between Plat-R primary vs. Plat-S
primary tumors (ICON dataset)

| | Mean Fold Change in | Up or Down in | | |
|---|---|---|---|---|
| Gene | Plat-R vs. Plat-S | Plat-R | P Value | FDR |
| FAP | 1.91002 | Up | 0.00197 | 0.30546 |
| (Gene | | | | |
| ID No. | | | | |
| 2191) | | | | |
| LOX | 1.55303 | Up | 0.00847 | 0.31350 |
| (Gene | | | | |
| ID No. | | | | |
| 4015) | | | | |
| MFAP2 | 1.55380 | Up | 0.00901 | 0.31350 |
| (Gene | | | | |
| ID No. | | | | |
| 4237) | | | | |
| MMP11 | 1.80984 | Up | 0.00846 | 0.31350 |
| (Gene | | | | |
| ID No. | | | | |
| 4320) | | | | |
| PGR | −1.98181 | Down | 0.00581 | 0.31350 |
| (Gene | | | | |
| ID No. | | | | |
| 5241) | | | | |
| PLVAP | 1.53990 | Up | 0.00220 | 0.30546 |
| (Gene | | | | |
| ID No. | | | | |
| 83483) | | | | |
| POSTN | 2.23263 | Up | 0.00669 | 0.31350 |
| (Gene | | | | |
| ID No. | | | | |
| 10631) | | | | |
| TIMP3 | 1.75621 | Up | 0.00286 | 0.30546 |
| (Gene | | | | |
| ID No. | | | | |
| 7078) | | | | |
| TP73 | −1.60160 | Down | 0.00030 | 0.21093 |
| (Gene | | | | |
| ID No. | | | | |
| 7161) | | | | |
| TSPAN8 | −2.13960 | Down | 0.00541 | 0.31350 |
| (Gene | | | | |
| ID No. | | | | |
| 7103) | | | | |

### Example 6

### POSTN Predicts Clinical Outcome of Front-Line Platinum-Based Chemotherapy in EOC

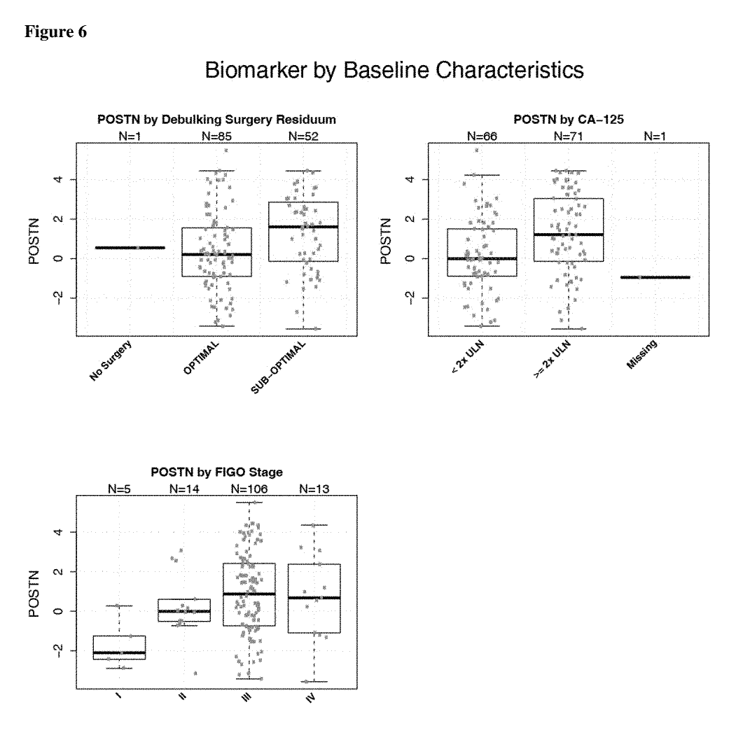

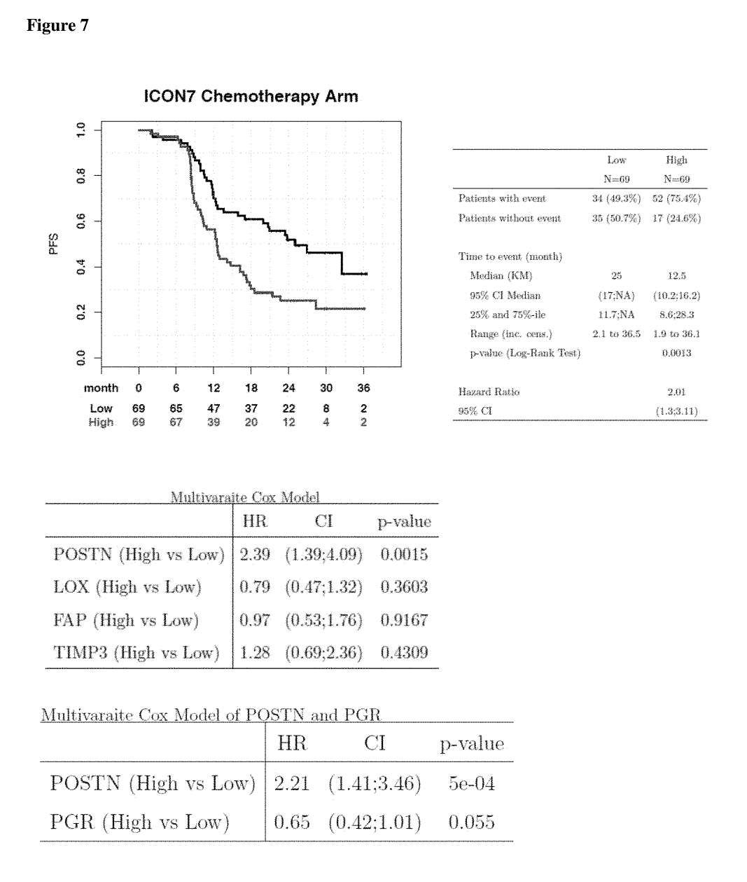

To examine whether the reactive stroma signature genes can predict clinical outcome of front-line chemotherapy in EOC, univariate survival analysis was performed on the chemo-control arm patients of the phase III trial using each of the four pre-specified reactive stroma signature genes, POSTN, FAP, TIMP3, and LOX, as well as PGR. As shown in FIG. 5B, patients with high POSTN expression (median cutoff) had significantly shorter progression free survival (PFS) with median PFS of 12 months compared to 27 months in patients with low POSTN expression (HR=2.4, 95% CI: 1.6-3.7, p=0.0001). Although weak correlation was observed between POSTN expression levels and several known clinical prognostic factors, including debulking status, serum CA125 level, and FIGO stages (FIG. 6), the association between POSTN levels and PFS remained significant (HR=1.76, p=0.015) after adjusting for these covariates. TIMP3 expression was also found to be significantly associated with PFS (HR=1.8, 95% CI: 1.2-2.8, p=0.0073) in the univariate Cox model (FIG. 5B). On the other hand, association between FAP or LOX expression and PFS using a median cutoff was not statistically significant, but highly significant when using a 75 percentile cutoff (HR=2.2, 95% CI: 1.4-3.4, p<0.001 for FAP; HR=1.9, 95% CI: 1.2-3.0, p=0.005 for LOX). Next, expression of all four genes (POSTN, FAP, LOX, and TIMP3) dichotomized using median cutoff was analyzed in a multivariate Cox regression model to assess the strength of association for each gene. Only expression of POSTN was significant in this multivariate analysis, suggesting that POSTN is the main driver and provides the predominant power for predicting patient outcome of front-line chemotherapy (FIG. 7). In addition, when expression of the four genes was averaged for each patient, the resulting overall stroma score did not improve association with PFS (HR=2.0, 95% CI: 1.3-3.1, p=0.0013), confirming POSTN's role as the defining stromal factor in predicting front-line ovarian cancer survival under chemotherapy. None of the signature genes showed significant association with overall survival (OS). To assess whether PGR provides additional predictive power of patient survival, multivariate COX model analysis was performed with dichotomized POSTN and PGR as covariates (FIG. 7). After adjusting for POSTN expression level, patients with higher PGR expression were found to experience a 35% decrease in risk of progression of ovarian cancer, however, the effect is only marginal, with a p value of 0.055 (HR=0.65, 95% CI: 0.42-1.01).

### Example 7

### Therapeutic Strategies to Overcome Chemotherapy-Resistance in Cancer

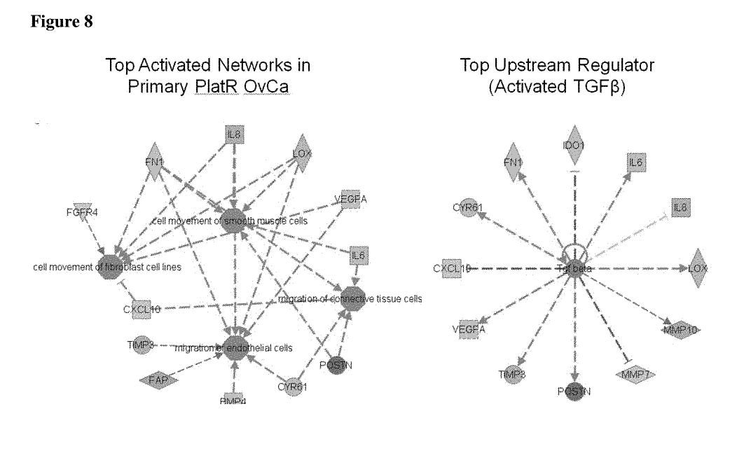

The specific association between reactive stroma, chemotherapy-resistance and poor clinical outcome identified from this study, highlighted the important interplay between cancer and the tumor microenvironment in ovarian cancer biology and treatment. Thus, targeting components of the tumor stroma in combination with agents directly targeting the tumor cells may provide a potential novel approach for overcoming resistance and improving efficacy. For example, POSTN can be one of the potential therapeutic targets. Up-regulation of POSTN has been observed in many cancer types, such as breast, lung, colon, pancreatic, and ovarian cancers. POSTN interacts with multiple cell-surface receptors, most notably integrins, and signals mainly via the PI3K/Akt and FAK-mediated pathways to promote cancer cell survival, angiogenesis, epithelial-mesenchymal transition (EMT), invasion, and metastasis. A recent study has demonstrated that stromal POSTN is crucial for metastatic colonization by regulating the interactions between breast cancer stem cells. Furthermore, targeting endogenous POSTN with a neutralizing antibody in an ovarian cancer cell line inhibited ovarian tumor growth and metastasis in animal models. Taken together, the important roles of POSTN in cancer development, progression and treatment response make it a promising novel therapeutic target for overcoming chemotherapy-resistance. In addition to individual stromal components, our study has revealed that the reactive stroma signature characterizing chemotherapy-resistance is highly enriched in genes involved in the normal process of wound healing. Consistent with previous experimental evidence, our data has suggested that TGF-β, a key mediator of the stromal response in wound repair, is likely to play an important role in regulating extensive cross-talks between tumor cells and their associated stroma (FIG. 8). Therefore, targeting TGF-β signaling pathway may be another potential promising therapeutic strategy for overcoming chemotherapy-resistance.

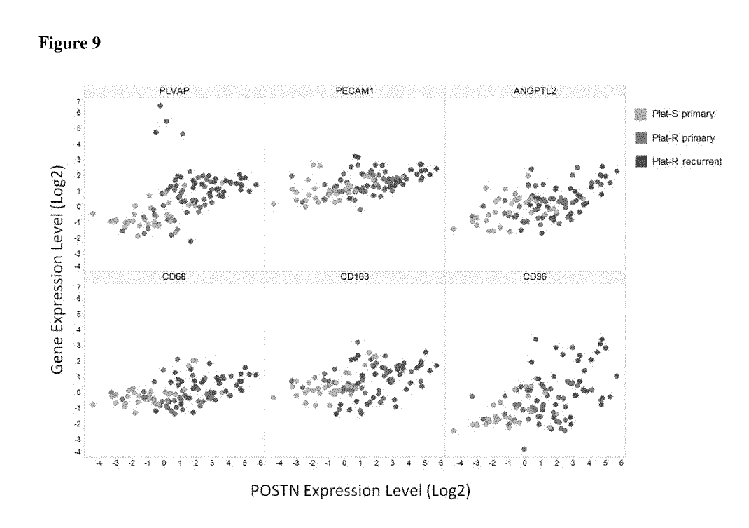

Analysis of genes whose expression levels are significantly correlated with the reactive stroma signature genes revealed other biological processes that may be involved in promoting chemotherapy-resistance. For examples, we found that POSTN expression level is highly correlated with PLVAP, PECAM1, and ANGPTL2, key components in promoting angiogenesis and vascular development (FIG. 9). Therefore, adding anti-angiogenesis reagents to the chemotherapy backbone, such as bevacizumab, may provide additional benefits to ovarian patients who are intrinsically resistant to primary chemotherapy. In addition, another therapeutic strategy for overcoming primary chemotherapy-resistance arose from the observation that POSTN expression level was highly correlated with CD68 and CD163, both are well-characterized surface markers of M2 macrophages known to be involved in inflammatory and immune responses during wound healing process (FIG. 9). This observation is consistent with a recent report that a stromal response expression signature is correlated with M2 macrophage infiltration and predict poor prognosis in gastric and ovarian cancer. Thus it is conceivable that anti-inflammatory drugs targeting M2 macrophages directly or the associated chemokines, cytokines, or growth factors, may represent another novel therapeutic strategy for overcoming primary chemotherapy-resistance in EOC.

### Example 8

### Circulating POSTN as a Marker to Predict Platinum-Resistant EOC

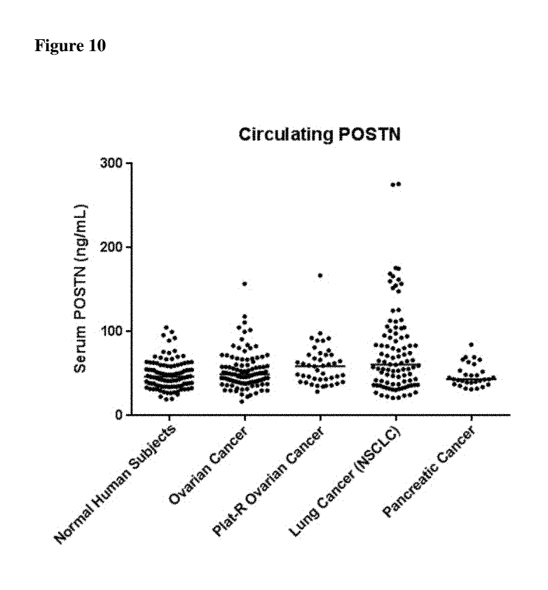

To investigate whether circulating POSTN could be used to predict chemoresistance in EOC patients, an ELISA assay was employed to measure circulating POSTN in serum. Serum POSTN levels were measured in vendor procured panels of serum samples from 102 age-matched normal healthy subjects (NHS), 100 EOC patients of unknown chemosensitivity, 43 EOC patients that are known to be platinum-resistant, 96 lung cancer (NSCLC) patients, and 29 pancreatic cancer patients. Chemosensitivity status and time of serum collection (before or after treatment) is unknown for the 100 vendor procured samples, however based on prevalence studies it is likely that at least 30% of the samples were from chemoresistant patients. The serum POSTN ELISA was sensitive down to 1.88 ng/mL and POSTN was detected in the serum of all the ovarian cancer patients and NHS. The grouped dot plot in FIG. 10 shows that the range of POSTN expression in the EOC patients was highly overlapping with that of NHS and with the other cancer patients. However, the median and range of circulating POSTN was significantly higher in both the chemoresistant ovarian cancer and NSCLC patients than NHS. These results are consistent with the tissue POSTN expression being higher in chemoresistant ovarian cancer patients.

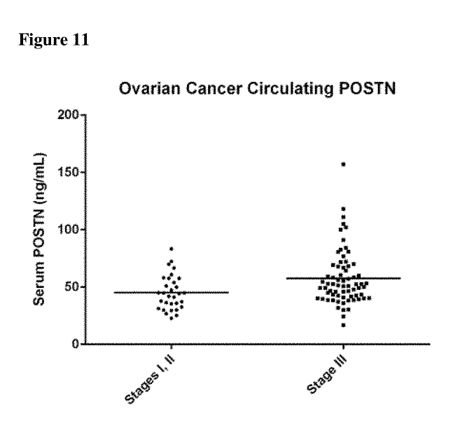

Circulating POSTN levels were also measured in vendor procured serum samples from stage I (25) and II (6) patients (31 combined) and 69 samples from stage III patients (as determined by FIGO Staging of Ovarian Cancer). A positive correlation was found between circulating POSTN and the stage of disease (FIG. 11). Based on these results, the measurement of circulating POSTN can also be used to as a non-invasive method to determine the stage of EOC patients.

Sequence Listing Key

| SEQ | |
|---|---|
| ID | |
| NO: | Sequence |
| 1 | QVHLQQSGAELAKPGASVHMSCKASGYTFTTYWMHWVKQRPGQGLE |
| | WIGYINPNTGYADYNQKFRDKATLTADKSSSTAYMQLSSLTSEDST |
| | VYFCARRRTGTSYFDYWGQGTTLTVSSTKTTPPSV |
| 2 | QTVLSQSPAILSASPGEKVTMTCRASSSVTYMHWYQQKPGSSPKPW |
| | IFATSNLASGVPARFSGSGSGTSYSLTISRVEAEDAATYYCQQWTS |
| | NPLTFGAGTK |
| 3 | QVQLQQSGAELARPGASVKLSCKASGYSFTHYWMQWVKQRPGQGLE |
| | WIGAIYPGDGDTRYTQRLKGKATLTADKSSSTAYMELSLASEDSAV |
| | YYCAREGEGNSAMDYWGQGTSVTVSSAKTTPPSV |
| 4 | DIVMTQSQKFMSTSVGDRVSVTCKASQNVGSSVAWFQQKPGQSPKT |
| | LIYSASYRDSGVPDRFTGSGSGTDFTLTITNVQSEDLTDYFCLQYG |
| | TYPYTFGGGTR |

Although the foregoing invention has been described in some detail by way of illustration and example for purposes of clarity of understanding, the descriptions and examples should not be construed as limiting the scope of the invention. The disclosures of all patents, patent applications, scientific references cited herein are expressly incorporated by reference in their entirety for all purposes as if each patent, patent application, scientific reference were specifically and individually incorporated by reference.

1. A method of identifying a patient with cancer that is chemotherapy-resistant, the method comprising
a) determining the expression level of one or more stroma signature gene(s) in a sample obtained from the patient,
b) comparing the expression level of the one or more stroma signature gene(s) to the median level of expression for the one or more stroma signature gene(s) in the cancer type, and
c) determining if the patient's cancer is chemotherapy-resistant, wherein expression of the one or more stroma signature gene(s) in the patient sample at a level more than the median level for expression of the one or more stroma signature gene(s) in the cancer type indicates that the patient has cancer that is chemotherapy-resistant.

2. The method of claim 1, wherein the patient has cancer that is chemotherapy-resistant if the patient's cancer has been determined to express the one or more stroma signature gene(s) at a level that is more than the 75^{th} percentile for the one or more stroma signature gene(s) expression in the cancer type.

3. The method of claim 1 or 2, wherein the one or more stroma signature gene is selected from the group consisting of POSTN, LOX, TIMP3, FAP, BGN, FGF1, FN1, ANGPTL2, ACTA2, MMP11, RBP4, CD36, PLVAP, PECAM1, GZMK, CD247, ABCC9, PCOLCE, CD1C, MS4A1, CD44, PMEPA1, IL7R, FBLN1, TWIST1, ID1, RAC2, GFRA1, CCR7, MAN1A1, EVI2A, PTPRC CD45RA, FCRLS, NNMT, CD27, SLA, TDO2, NUAK1, and COL4A1.

4. The method of claim 3, wherein the stroma signature gene is POSTN.

5. The method of claim 3, wherein the one or more stroma signature gene(s) is POSTN and FAP; POSTN and TIMP3; POSTN and LOX; POSTN, FAP, and TIMP3; POSTN, FAP, and LOX; POSTN, TIMP3, and LOX; or POSTN, FAP, TIMP3, and LOX.

6. The method of any one of claims 1-5, wherein the sample is a tumor tissue sample, a blood sample, or a serum sample.

7. The method of any one of claims 1-6, wherein the cancer that is chemotherapy-resistant is cancer that is platinum-resistant.

8. The method of any one of claims 1-7, wherein the method is carried out prior to administering a chemotherapeutic agent in order to provide a pre-administration diagnosis.

9. The method of any one of claims 1-7, wherein the patient has not undergone chemotherapy or wherein the patient is currently undergoing chemotherapy.

10. The method of any one of claims 1-9, further comprising the step of identifying the patient as likely to benefit from administration of a VEGF antagonist when the patient is determined to have cancer that is chemotherapy-resistant.

11. The method of any one of claims 1-10, further comprising the step of administering a VEGF antagonist in a therapeutically effective amount to the patient, if the patient is determined to have cancer that is chemotherapy-resistant.

12. The method of claim 11, wherein the VEGF antagonist is an anti-VEGF antibody.

13. The method of claim 12, wherein the anti-VEGF antibody is bevacizumab.

14. The method of any one of claims 1-13, further comprising the step of identifying the patient as likely to benefit from a stroma-targeted therapy when the patient is determined to have cancer that is chemotherapy-resistant.

15. The method of any one of claims 1-14, further comprising the step of administering a stroma-targeted agent in a therapeutically effective amount to the patient, if the patient is determined to have a cancer that is chemotherapy-resistant.

16. The method of claim 15, wherein the stroma-targeted agent is an anti-periostin (POSTN) antibody.

17. The method of any one of claims 1-16, further comprising the step of identifying the patient as likely to benefit from an immunotherapy when the patient is determined to have cancer that is chemotherapy-resistant.

18. The method of any one of claims 1-17, further comprising the step of administering an immunomodulatory agent in a therapeutically effective amount to the patient, if the patient is determined to have cancer that is chemotherapy-resistant.

19. The method of claim 18, wherein the immunomodulatory agent comprises a TDO2, CD36, GZMK, CD247, CD1C, CSF1R, IDO1, IL7R, or CCR7 antagonist.

20. The method of any one of claims 1-19, wherein the cancer is primary, advanced, refractory, or recurrent.

21. The method of any one of claims 1-20, wherein the cancer is a gynecologic cancer selected from the group consisting of ovarian cancer, peritoneal cancer, fallopian tube cancer, cervical cancer, endometrial cancer, vaginal cancer, and vulvar cancer.

22. The method of claim 21, wherein the gynecologic cancer is ovarian cancer.

23. The method of any one of claims 1-20, wherein the cancer is selected from the group consisting of colorectal cancer, breast cancer, non-small cell lung cancer (NSCLC), kidney cancer (renal cell carcinoma), or brain cancer (glioblastoma).

24. A method of identifying a patient with cancer that is chemotherapy-sensitive, the method comprising
a) determining the expression level of one or more stroma signature gene(s) in a sample obtained from the patient,
b) comparing the expression level of the one or more stroma signature gene(s) to the median level of expression for the one or more stroma signature gene(s) in the cancer type, and
c) determining if the patient has cancer that is chemotherapy-sensitive, wherein expression of the one or more stroma signature gene(s) in the patient sample at a level less than the median level for expression of the one or more stroma signature gene(s) in the cancer type indicates that the patient has cancer that is chemotherapy-sensitive.

25. The method of claim 24, wherein the patient has cancer that is chemotherapy-sensitive if the patient's cancer has been determined to express the one or more stroma signature gene(s) at a level that is less than the 25^{th} percentile for the one or more stroma signature gene(s) expression in the cancer type.

26. The method of claim 24 or 25, wherein the one or more stroma signature gene is selected from the group consisting of POSTN, LOX, TIMP3, FAP, BGN, FGF1, FN1, ANGPTL2, ACTA2, MMP11, RBP4, CD36, PLVAP, PECAM1, GZMK, CD247, ABCC9, PCOLCE, CD1C, MS4A1, CD44, PMEPA1, IL7R, FBLN1, TWIST1, ID1, RAC2, GFRA1, CCR7, MAN1A1, EVI2A, PTPRC/CD45RA, FCRLS, NNMT, CD27, SLA, TDO2, NUAK1, and COL4A1.

27. The method of claim 26, wherein the stroma signature gene is POSTN.

28. The method of claim 26, wherein the one or more stroma signature gene(s) is POSTN and FAP; POSTN and TIMP3; POSTN and LOX; POSTN, FAP, and TIMP3; POSTN, FAP, and LOX; POSTN, TIMP3, and LOX; or POSTN, FAP, TIMP3, and LOX.

29. The method of any one of claims 24-28, wherein the sample is a tumor tissue sample, a blood sample, or a serum sample.

30. The method of any one of claims 24-29, further comprising the step of administering one or more chemotherapeutic agent(s) in a chemotherapy regimen, if the patient is determined to have cancer that is chemotherapy-sensitive.

31. The method of claim 30, wherein the one or more chemotherapeutic agent(s) is selected from the group consisting of a HER antibody, an antibody directed against a tumor associated antigen, an anti-hormonal compound, a cardioprotectant, a cytokine, an EGFR-targeted drug, an anti-angiogenic agent, a tyrosine kinase inhibitor, a COX inhibitor, a non-steroidal anti-inflammatory drug, a farnesyl trasferase inhibitor, an antibody that binds oncofetal protein CA 125, a Her2 vaccine, a HER targeting therapy, a raf or ras inhibitor, liposomal doxorubicin, topotecan, taxane, dual tyrosine kinase inhibitor, TLK286, EMD-7200, a medicament that treats nausea, a medicament that prevents or treats skin rash or standard acne therapy, a medicament that treats or prevents diarrhea, a body temperature-reducing medicament, and a hematopoietic growth factor.

32. The method of claim 30, wherein the one or more chemotherapeutic agent(s) is gemcitabine, carboplatin, oxaliplatin, irinotecan, fluoropyrimidine (e.g., 5-FU), paclitaxel (e.g., nab-paclitaxel), docetaxel, topotecan, capecitabine, lecovorin, temozolomide, interferon-alpha, or liposomal doxorubicin (e.g., pegylated liposomal doxorubicin).

33. The method of claim 30, wherein the chemotherapy regimen comprises the administration of carboplatin and paclitaxel; carboplatin and gemcitabine; or paclitaxel, topotecan, or pegylated liposomal doxorubicin.

34. The method of claim 30, wherein the chemotherapy regimen comprises the administration of capecitabine and paclitaxel; or capecitabine and docetaxel.

35. The method of claim 30, wherein the chemotherapy regimen comprises the administration of temozolomide and optionally radiotherapy.

36. The method of claim 30, wherein the chemotherapy regimen comprises the administration of fluropyrimidine, irinotecan, cisplatin, fluropyramidine and oxaliplatin; fluropyrimidine and irinotecan; fluropyramidine, lecovorin, and oxaliplatin; or ironotecan, fluoropyrimidine, and leucovorin.

37. The method of claim 30, wherein the chemotherapy regimen comprises the administration of paclitaxel and topotecan; or paclitaxel and cisplatin.

38. The method of claim 30, wherein the chemotherapy regimen comprises the administration of interferon-alpha2a.

39. The method of any one of claims 1-19, wherein the cancer is primary, advanced, refractory, or recurrent.

40. The method of any one of claims 24-39, wherein the cancer is a gynecologic cancer selected from the group consisting of ovarian cancer, peritoneal cancer, fallopian tube cancer, cervical cancer, endometrial cancer, vaginal cancer, and vulvar cancer.

41. The method of claim 40, wherein the gynecologic cancer is ovarian cancer.

42. The method of any one of claims 24-39, wherein the cancer is selected from the group consisting of colorectal cancer, breast cancer, non-small cell lung cancer (NSCLC), kidney cancer (renal cell carcinoma), or brain cancer (glioblastoma).

43. A method of identifying a patient suffering from cancer who may benefit from administration of a VEGF antagonist or an immuno-modulatory agent, the method comprising:
a) determining the expression level of one or more stroma signature gene(s) in a sample obtained from the patient, wherein expression of the one or more stroma signature gene(s) at a level more than the median level for expression of the one or more stroma signature gene(s) in the cancer type indicates that the patient may benefit from administration of a VEGF antagonist or immunomodulatory agent, and optionally
b) administering the VEGF antagonist or immunomodulatory agent in a therapeutically effective amount to the patient.

44. The method of claim 43, wherein the VEGF antagonist is an anti-VEGF antibody.

45. The method of claim 44, wherein the anti-VEGF antibody is bevacizumab.

46. The method of claim 43, wherein the immunomodulatory agent comprises a TDO2, CD36, GZMK, CD247, CD1C, CSF1R, IDOL IL7R, or CCR7 antagonist.

47. A method of treating a patient with cancer, the method comprising administering to the patient a therapeutically effective amount of a stroma-targeted agent, wherein the patient's cancer has been determined to express one or more stroma signature gene(s) at a level more than the median level for expression of the one or more stroma signature gene(s) in the cancer type.

48. The method of any one of claims 43-47, further comprising administering one or more chemotherapeutic agent(s) to the patient.

49. The method of any one of claims 43-48, wherein the patient's cancer has been determined to express the one or more stroma signature gene(s) at a level that is more than the 75^{th} percentile for the one or more stroma signature gene(s) expression in the cancer type.

50. The method of any one of claims 43-49, wherein the one or more stroma signature gene(s) is selected from the group consisting of POSTN, LOX, TIMP3, FAP, BGN, FGF1, FN1, ANGPTL2, ACTA2, MMP11, RBP4, CD36, PLVAP, PECAM1, GZMK, CD247, ABCC9, PCOLCE, CD1C, MS4A1, CD44, PMEPA1, IL7R, FBLN1, TWIST1, ID1, RAC2, GFRA1, CCR7, MAN1A1, EVI2A, PTPRC CD45RA, FCRLS, NNMT, CD27, SLA, TDO2, NUAK1, and COL4A1.

51. The method of claim 50, wherein the stroma signature gene is POSTN.

52. The method of claim 50, wherein the one or more stroma signature gene(s) is POSTN and FAP; POSTN and TIMP3; POSTN and LOX; POSTN, FAP, and TIMP3; POSTN, FAP, and LOX; POSTN, TIMP3, and LOX; or POSTN, FAP, TIMP3, and LOX.

53. The method of any one of claims 43-52, wherein the cancer is chemotherapy-resistant, chemotherapy-sensitive, primary, advanced, refractory, or recurrent.

54. The method of any one of claims 43-53, wherein the cancer is a gynecologic cancer selected from the group consisting of ovarian cancer, peritoneal cancer, fallopian tube cancer, cervical cancer, endometrial cancer, vaginal cancer, and vulvar cancer.

55. The method of claim 54, wherein the gynecologic cancer is ovarian cancer.

56. The method of any one of claims 43-53, wherein the cancer is selected from the group consisting of colorectal cancer, breast cancer, non-small cell lung cancer (NSCLC), kidney cancer (renal cell carcinoma), or brain cancer (glioblastoma).

57. A method of determining the stage of ovarian cancer in a patient, the method comprising determining the expression level of POSTN in a sample obtained from the patient, wherein detection of an increased level of expression of POSTN in the patient sample, relative to a control, indicates an advanced stage of ovarian cancer.

58. The method of claim 57, wherein said control is the median level of POSTN expression in a population of patients having ovarian cancer.

59. The method of claim 57, wherein said control is the median level of POSTN expression in a population of patients having FIGO stage I or FIGO stage II ovarian cancer.

60. The method of claim 57, further comprising the step of administering a therapy to the patient, if the patient is determined to have ovarian cancer that is in the advanced stage.

61. The method of claim 57, wherein ovarian cancer in the advanced stage is FIGO Ovarian Cancer Stage III or IV.

62. The method of any one of claims 57-61, wherein the sample is a tumor tissue sample, a blood sample, or a serum sample.


SEQUENCE LISTING (4 sequences)

SEQ ID NO: 1 - protein, 127 aa, Mus musculus
QVHLQQSGAELAKPGASVHMSCKASGYTFTTYWMHWVKQRPGQGLEWIGYINPNTGYADYNQKFRDKATLTADKSSSTAYMQLSSLTSEDSTVYFCARRRTGTSYFDYWGQGTTLTVSSTKTTPPSV

SEQ ID NO: 2 - protein, 102 aa, Mus musculus
QTVLSQSPAILSASPGEKVTMTCRASSSVTYMHWYQQKPGSSPKPWIFATSNLASGVPARFSGSGSGTSYSLTISRVEAEDAATYYCQQWTSNPLTFGAGTK

SEQ ID NO: 3 - protein, 127 aa, Mus musculus
QVQLQQSGAELARPGASVKLSCKASGYSFTHYWMQWVKQRPGQGLEWIGAIYPGDGDTRYTQRLKGKATLTADKSSSTAYMELSSLASEDSAVYYCAREGEGNSAMDYWGQGTSVTVSSAKTTPPSV

SEQ ID NO: 4 - protein, 103 aa, Mus musculus
DIVMTQSQKFMSTSVGDRVSVTCKASQNVGSSVAWFQQKPGQSPKTLIYSASYRDSGVPDRFTGSGSGTDFTLTITNVQSEDLTDYFCLQYGTYPYTFGGGTR